(19) United States
(12) Patent Application Publication
(10) Pub. No.: US 2008/0064965 A1
(43) Pub. Date: Mar. 13, 2008

(54) Devices and methods for measuring pulsus paradoxus

(76) Inventors: Gregory D. Jay, Norfolk, MA (US); Megan Wachs, Elkridge, MD (US); Devraj Banerjee, Brooklyn, NY (US)

Correspondence Address: CLARK & ELBING LLP, 101 FEDERAL STREET, BOSTON, MA, 02110, US

(21) Appl. No.: 11/899,512

(22) Filed: Sep. 6, 2007

Related U.S. Application Data
(60) Provisional application No. 60/843,307, filed on Sep. 8, 2006.

(51) Int. Cl. A61B 5/0205; A61B 5/08

(52) U.S. Cl. 600/484; 600/529

(57) ABSTRACT

The invention relates to methods and devices for measuring pulsus paradoxus. The methods herein employ a combination of one or more forms of waveform analysis for the purpose of measuring pulsus paradoxus and diagnosing respiratory distress. The methods also combine measurements of pulsus paradoxus and physician assessments to diagnose respiratory distress. The methods also combine measurements of pulsus paradoxus and percentage oxygenated hemoglobin to diagnose respiratory distress. The devices of this invention employ pulse oximeters, arterial tonometers, finometers, or processors for the purpose of implementing the methods of the invention.

### CROSS-REFERENCE TO RELATED APPLICATIONS

This application claims benefit of U.S. Provisional Application No. 60/843,307, filed Sep. 8, 2006, which is hereby incorporated by reference.

### BACKGROUND OF THE INVENTION

The medical term pulsus paradoxus refers to a quantifiable, exaggerated decrease in arterial blood pressure during inspiration. In normal subjects, the decrease in arterial blood pressure during inspiration is in the range of about 2-5 mm Hg; whereas, in a subject suffering from certain medical conditions, pulsus paradoxus during inspiration may exceed this range and be on the order of 5-20 mm Hg or higher. The National Asthma Education and Prevention Program Expert Panel Report 1 (NAEPP EPR1) guidelines in 1991 specified 12 mmHg as the pulsus paradoxus level which supported hospital admission. Pulsus paradoxus has been noted in a variety of medical conditions including, but not limited to, asthma, croup, tension pneumothorax, pericardial tamponade, pericardial effusions, pulmonary embolus, hypovolemic shock, and sleep apnea.

Pulsus paradoxus is a function of the underlying disease process. In severe acute asthma, for example, large intrathoracic pressure variations are created by air trapping, causing a net increase in intraluminal airway pressure. The increased airway pressure is mechanically translated into increased intrapleural pressure, from a dramatically negative intrapleural pressure level during inspiration, to a positive intrapleural pressure level during expiration. Elevated intrathoracic pressure translates to increased impedance to right ventricular ejection which causes a markedly impaired left ventricular stroke output and concomitant reduction of left ventricular preload. Similar alterations contribute to paradoxic pulse in other respiratory and cardiovascular disease states.

Pulsus paradoxus has been a cornerstone in the evaluation of subjects with acute asthma. The value of pulsus paradoxus as a pathophysiologic measure is well established. For example, in a prospective clinical study of 85 asthmatic children, it was reported that a pulsus paradoxus measurement of 11 mm Hg differentiated those children who needed hospitalization from those who did not. However, measurement of pulsus paradoxus is rarely performed and accuracy of its measurement via sphygmomanometry is questionable. Resistance by physicians to the application of pulsus paradoxus for the objective assessment of disease severity, asthma in particular, is largely due to the difficulty in measuring pulsus paradoxus in a rapidly breathing subject by methods currently employed. Despite this, pulsus paradoxus has been used in a number of asthma studies and continues to be a recommended metric by the NAEPP Expert Panel Report 2.

One conventional method for measuring pulsus paradoxus in a hospital emergency room setting is by the application of a sphygmomanometer, commonly referred to as a blood pressure cuff, that is cyclically inflated/deflated near a subject's systolic blood pressure. The operator determines systolic pressure during inspiration and expiration in separate maneuvers. This requires simultaneous observation of respiratory phase and cuff pressure. Typically, multiple operator efforts are required in order to arrive at a systolic pressure during inspiration and expiration. The objective is to determine how much the subject's blood pressure decreases during inspiration by bracketing the decrease in blood pressure within the cyclically varied cuff pressure. This process is ergonomically very difficult to perform and made even more so by the rapidly breathing subject. As a result, the method is inaccurate and inter-observer results are excessively variable.

Other measures used currently to assess the severity of asthma are clinical assessment, arterial blood gas analysis, spirometry, arterial tonometry, pulse oximetry; however, all are subject to certain shortcomings. Clinical assessment scores, for example, exhibit marked inter-observer variability and have been incompletely validated. Arterial blood gas analysis is an invasive and painful technique and is often complicated by therapeutic administration of O₂ and β-adrenergic drugs and is therefore unreliable as an indicator of asthma severity. Tests of forced expiratory flow, as in spirometry, are effort dependent, typically cannot be used with children, and may actually exacerbate the underlying disease process. Pulse oximetry has been used to estimate pulsus paradoxus, but potential methods of interpreting pulse oximetry data to measure pulsus paradoxus with even greater accuracy have not been fully explored.

Many experts are stymied to explain the rising mortality of asthmatic subjects in view of the improving quality of acute pharmacological management of asthma and the enhanced sophistication of emergency physicians, as well as pre-hospital care systems. One explanation lies in the observation that there has been little change in how the asthmatic subject is evaluated acutely. An effort-independent, non-invasive, and highly accurate measurement of pulsus paradoxus that provides immediate insight into how troubled is the act of breathing would be invaluable in the emergency room setting or home monitoring.

Thus, a need exists for an objective criterion in evaluating pulsus paradoxus, which is independent of effort, accurate, and familiar to clinicians.

### SUMMARY OF THE INVENTION

The invention relates to methods and devices for measuring pulsus paradoxus. The methods herein employ a combination of one or more forms of waveform analysis for the purpose of measuring pulsus paradoxus and diagnosing respiratory distress. The devices of this invention employ pulse oximeters, arterial tonometers, or other blood pressure-monitoring instruments and processors for the purpose of implementing the methods of the invention.

In one embodiment, the invention features a method for measuring pulsus paradoxus in a subject including collecting pulsatile cardiorespiratory data, e.g., a plethysmographic waveform obtained from a pulse oximeter, an arterial tonometer, or a finometer, from the subject; performing period amplitude analysis on the data; performing power spectrum analysis on the data; and combining the analyses to determine a measurement for pulsus paradoxus. The method may further include comparing the measurement for pulsus paradoxus in the subject to that obtained in a healthy subject, wherein a determination that the measurement for the subject exceeds the measurement for the healthy subject by at least 10%, e.g., a difference in blood pressure measured in mmHg, indicates the subject is experiencing respiratory distress. The data may be collected from the subject over the course of a time interval, e.g., of at least 30 seconds, at least 60 seconds, or at least 2 minutes. The data may be filtered using a bandpass filter, e.g., a bandpass filter that substantially excludes signal frequencies less than 3 times the frequency of respiration of the subject or signal frequencies greater than 7 times the frequency of respiration of the subject. The period amplitude analysis may include a determination of the maximum difference in height of any two peaks, the maximum difference in area under any two peaks, the maximum difference in slope of any two peaks, the maximum difference in curve length of any two peaks present in the data, the average maximum difference in height of any two peaks, the average maximum difference in area under any two peaks, the average maximum difference in slope of any two peaks, or the average maximum difference in curve length of any two peaks present in the data. The period amplitude analysis may be further converted into a change in blood pressure associated with pulsus paradoxus, e.g., a change of at least 10, 11, or 12 mmHg indicating respiratory distress and motivating medical admission of a subject. The period amplitude analysis may be converted using a transfer function, e.g., a transfer function of 0.01 Volts/mmHg, determined from data of subjects experiencing respiratory distress, e.g., respiratory distress caused by asthma or artificial means. The period amplitude analysis may be compared with period amplitude analysis determined using pulsatile cardiorespiratory data from healthy subjects or subjects experiencing respiratory distress, e.g., respiratory distress caused by asthma or by artificial means, wherein, e.g., the comparing yields a difference in blood pressure measured in mmHg. The power spectrum analysis may include a determination of signal amplitude, e.g., an average signal amplitude, associated with respiration present in the data. The power spectrum analysis may be converted to a change in blood pressure associated with pulsus paradoxus, e.g., a change in blood pressure at least 10, 11, or 12 mmHg indicating respiratory distress and motivating medical admission of a subject, using a transfer function, e.g., a quadratic function, determined from data of subjects experiencing respiratory distress, e.g., respiratory distress caused by asthma or by artificial means. The power spectrum analysis may be compared with power spectrum analysis determined using pulsatile cardiorespiratory data from healthy subjects or subjects experiencing respiratory distress, e.g., respiratory distress caused by asthma or artificial means, wherein, e.g., the comparing yields a difference in blood pressure measured in mmHg. The combining step may include converting the period amplitude analysis and the power spectrum analysis into changes in blood pressure associated with pulsus paradoxus, e.g., changes in blood pressure at least 10, 11, or 12 mmHg indicating respiratory distress and motivating medical admission of a subject or changes in blood pressure between 5 mmHg and 11 mmHg motivating medical monitoring of a subject, and averaging those changes, calculating a moving average of those changes, calculating a Kappa statistic relating those changes, or calculating a test statistic that determines whether the smaller of the two changes in blood pressure is at least 50% of the size of the larger of the two changes in blood pressure.

In an alternate embodiment, the invention features a method for measuring pulsus paradoxus including collecting pulsatile cardiorespiratory data from the subject; performing a first form of waveform analysis on the data; performing a second form of waveform analysis on the data; and combining the analyses to determine a measurement for pulsus paradoxus, e.g., combining the analyses with a third form of waveform analysis performed on the data to measure pulsus paradoxus.

In another embodiment, the invention features a device for measuring pulsus paradoxus in a subject including an optical plethysmograph, e.g., a pulse oximeter, to collect pulsatile cardiorespiratory data from the subject; a processor to perform period amplitude analysis on the data; a processor to perform power spectrum analysis on the data; and a processor to combine the analyses to determine a measurement for pulsus paradoxus. The device may also include a bandpass filter to filter the data, e.g., a bandpass filter that substantially excludes signal frequencies less than 3 times the frequency of respiration of the subject or signal frequencies greater than 7 times the frequency of respiration of the subject.

In another embodiment, the invention features a device for measuring pulsus paradoxus in a subject including an arterial tonometer to collect pulsatile cardiorespiratory data from the subject; a processor to perform period amplitude analysis on the data; a processor to perform power spectrum analysis on the data; and a processor to combine the analyses to determine a measurement for pulsus paradoxus. The device may also include a bandpass filter to filter the data, e.g., a bandpass filter that substantially excludes signal frequencies less than 3 times the frequency of respiration of the subject or signal frequencies greater than 7 times the frequency of respiration of the subject.

In an alternate embodiment, the invention features a device for measuring pulsus paradoxus in a subject including a finometer to collect pulsatile cardiorespiratory data from the subject; a processor to perform period amplitude analysis on the data; a processor to perform power spectrum analysis on the data; and a processor to combine the analyses to determine a measurement for pulsus paradoxus. The device may also include a bandpass filter to filter the data, e.g., a bandpass filter that substantially excludes signal frequencies less than 3 times the frequency of respiration of the subject or signal frequencies greater than 7 times the frequency of respiration of the subject.

In another embodiment, the invention features a device for measuring respiratory distress in a subject including an optical plethysmograph, e.g., a pulse oximeter, to collect pulsatile cardiorespiratory data from the subject; a processor to calculate pulsus paradoxus from the data; a processor to calculate percentage oxygenated hemoglobin from the data; and a processor to combine calculation outputs to determine a measurement of respiratory distress.

In a final embodiment, the invention features a method for measuring respiratory distress in a subject including collecting pulsatile cardiorespiratory data from the subject; estimating pulsus paradoxus using the data; estimating the percentage of hemoglobin (Hb) which is saturated with oxygen; and combining the analyses to determine a measurement of respiratory distress.

“Component of a signal or waveform” as used herein means a part of a given signal or waveform having a given frequency, typically measured in Hertz (Hz). The given signal or waveform may have one or more components and the given signal or waveform is considered to be the sum of its components.

“Exceeds” as used herein means two unequal numbers having a non-zero difference, a factor increase, or a factor decrease between them. For example, one number exceeds another if one of those numbers is at least 10%, at least 20%, at least 30%, at least 40%, at least 50%, at least 60%, at least 70%, at least 80%, at least 90%, or at least 200% greater than or smaller than the other. Alternatively, for example, one number exceeds another if it is 1.5, 2, 3, 4, 5, 6 or more times larger or smaller than another number. A first number, for example, may exceed a second number if the first number is larger than the second number. A first number, for example, may exceed a second number if the first number is smaller than the second number.

“Peak” as used herein means the curved region of a waveform, such as, e.g., a waveform created by continuous monitoring of a pulse, approximately centered around a local maximum of that waveform and extending to the closest local minima on either side of that local maximum. A waveform, typically depicted on a two-dimensional graph having a x-axis and a y-axis, will contain a series of peaks, often at regular intervals, and a single peak is typically identified as the curved region between two adjacent local minima along a waveform. The “area under” a peak is the area contained by the closed region defined by the boundaries of that peak and a diagonal or a horizontal baseline, such as, e.g., the x-axis. The “height” of a peak is the vertical distance between the local maximum of a peak and a diagonal or horizontal baseline below that local maximum. The “curve length” of a peak is the sum of the amplitude changes along the peak waveform. The “slope” of a peak is the ratio of the curve length of the peak to the period of the peak, i.e., the horizontal distance between the local minima that form the boundaries of a peak. A complete description of peaks, i.e., half-waves, are described in Feinberg et al. Electroencephalography and Clinical Neurophysiology 44:202-213, 1978.

“Period amplitude analysis” or “periodic amplitude analysis” as used herein means a form of waveform analysis that involves the comparison of features of two or more peaks along a given waveform. For example, comparisons of two or more peaks performed using period amplitude analysis may include comparing the features of those peaks, such as the peaks' periods, heights (i.e., amplitudes), areas under the peaks (i.e., integrated amplitudes), curve lengths, slopes, average heights, average areas under the peaks, average curve lengths, average slopes, frequency of the waveform components, or the maximum of any of these features. Typically, differences in one or more features of two or more peaks is indicative of a perturbation of the waveform, such as, e.g., the periodic attenuation of a pulsatile cardiorespiratory waveform caused by pulsus paradoxus. Various forms of period amplitude analysis are known in the art and are described in Feinberg et al. Electroencephalography and Clinical Neurophysiology 44:202-213, 1978; Uchida et al. Physiology & Behavior 67:121-131, 1999; Borbely et al. “Processes Underlying Sleep Regulation.” Psychopharmacology 2000; Cantero et al. Journal on Neuroscience 22:4702-4708, 2002; Armitage et al. Curr. Rev. Mood Anxiety Disord. 1: 139-51, 1997; Nunez Electrical fields of the brain. New York: Oxford Press; 1981; Hoffmann et al. Waking Sleeping 3:1-16, 1979; Armitage et al. Biol Psychiatry 31:52-68, 1992.

“Plethysmographic waveform” as used herein means the waveform derived from blood pressure. For example, a plethysmographic waveform can be established by monitoring a subject's arterial blood pressure using, e.g., a pulse oximeter or an arterial tonometer. A plethysmographic waveform may contain peaks, local maxima, and local minima upon which various forms of waveform analysis may be performed.

“Power spectrum analysis” as used herein means a form of waveform analysis that involves decomposition of a waveform into its composite sinusoidal waveforms (including cosine waveforms), each having a characteristic frequency, and identification of the corresponding amplitudes associated with its sinusoidal waveform components. “Amplitudes” or “signal amplitudes” as used herein refer to the strength or intensity of a signal, a waveform, or a sinusoidal waveform component; waveform components with large amplitudes are stronger than components with small amplitudes. Sinusoidal waveforms that make larger contributions to the original waveform will have larger amplitudes as calculated by power spectrum analysis, and sinusoidal waveforms that make no contribution to the original waveform will have amplitudes of zero. Various related mathematical techniques known in the art can be used to generate a power spectrum of a waveform; some exemplary techniques are Fourier decomposition or Fourier transformation, Discrete Fourier Transformation, Fast Fourier Transformation, Z-transformation, Fractional Fourier Transformation, Welch's method, and the maximum entropy method (Bracewell, The Fourier Transform and Its Applications, 3rd ed. New York: McGraw-Hill, 1999; Brigham, The Fast Fourier Transform and Applications. Englewood Cliffs, N.J.: Prentice Hall, 1988). Once generated, the power spectrum can be used to identify different signals embedded in the original waveform, for example, a signal associated with heart beat and a signal associated with respiration.

“Pulsatile cardiorespiratory data” as used herein means data that measures blood pressure (pulse), or respiration of a subject, or both. Such data may be obtained from a single source, such as a pulse oximeter or an arterial tonometer, or multiple sources. Pulsatile cardiorespiratory data may include a plethysmographic waveform. Waveform analysis may be applied to a plethysmographic waveform in order to identify components of the waveform associated with different signals, for example, decomposing data collected by a pulse oximeter or an arterial tonometer into a signal associated with heart beat and a signal associated with respiration.

“Respiratory distress” as used herein means the physical condition and symptoms caused by an obstructed airway due to a medical condition, such as, e.g., pneumonia, respiratory tract infection, asthma, allergic reaction, croup, tension pneumothorax, pericardial tamponade, pericardial effusions, pulmonary embolus, hypovolemic shock, and sleep apnea, or artificial means, such as that caused by an obstructed breathing apparatus employed in various medical studies. Exemplary symptoms of respiratory distress include tachypnea, expiratory wheezing, inspiratory wheezing, silent chest, accessory muscle use, audible wheezing, paradoxical respirations, and respiratory failure.

“Substantially excludes pulse frequencies” as used herein means the exclusion of a majority of pulse frequencies, e.g., 50%, 60%, 70%, 80%, 90%, or 99% of frequencies, outside of the permitted range. For example, a bandpass filter may be used to substantially exclude pulse frequencies below a first cutoff frequency and above a second cutoff frequency, such that frequencies between the first and second cutoff frequencies are permitted.

“Transfer function” as used herein means a mathematical function that converts a given number to another number. The given number can have units, no units, or arbitrary units, and can be converted to a number with a different type of units, arbitrary units, or presence of units. For example, a measurement of a number of volts, when converted by a transfer function, e.g., a ratio or a quadratic function, is converted to a number in units of mm Hg, indicating the blood pressure associated with that number of volts.

“Waveform analysis” as used herein means any mathematical technique that analyzes and/or quantifies the shape, geometry, periodicity, composition, distribution, or patterns of one or more waveforms, e.g., a plethysmographic waveform. Exemplary forms of waveform analysis include, without limitation, period amplitude analysis, power spectrum analysis, and singular value decomposition.

### BRIEF DESCRIPTION OF THE DRAWINGS

FIG. 1 is a photograph of a pulsus paradoxus (PP) monitor setup consisting of laptop computer equipped with an analog to digital conversion interface and a continuous non-invasive blood pressure monitor.

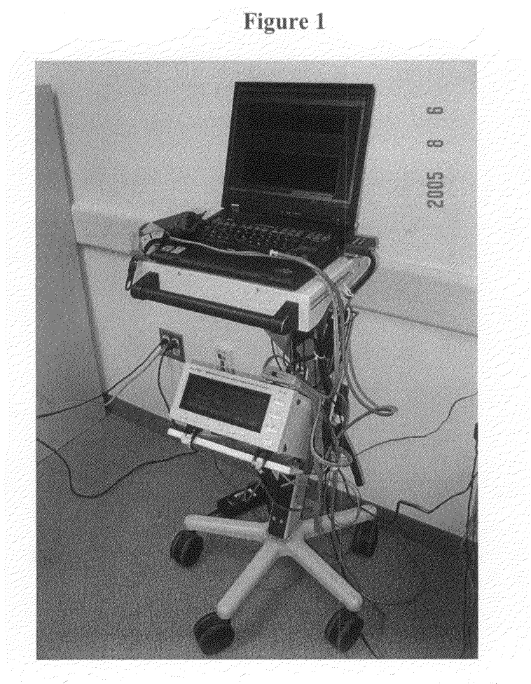

FIG. 2A depicts a plot of automated-pulsus paradoxus (AT-PP) sensitivity and specificity as a function of PP threshold in asthma disposition during post-treatment. The PP threshold, which maximized sensitivity and specificity, is identified. Cost of care is illustrated in the right hand axis. (Inserts). Corresponding receiver operator curves where the symbol ‘*’ denotes the sensitivity and specificity, which maximized area under the curve.

FIG. 2B depicts a plot of automated-pulsus paradoxus (AT-PP) sensitivity and specificity as a function of PP threshold in asthma disposition during pre-treatment. The PP threshold, which maximized sensitivity and specificity, is identified. Cost of care is illustrated in the right hand axis. (Inserts). Corresponding receiver operator curves where the symbol ‘*’ denotes the sensitivity and specificity, which maximized area under the curve.

FIG. 3 depicts a Bland and Altman plot of respiratory rate measured by trained bedside observers compared to predicted respiratory rate from the AT-PP monitor.

FIG. 4A depicts representative PP data from a blood pressure monitor (FINAPRES) and oximetry plethysmograph recorded simultaneously. Arrows indicates a maxima and minima systolic blood pressure induced by −20 mmHg inspiratory pressure, identified by the PP algorithm. Arrowheads denote corresponding plethysmograph waveforms, which also indicate the presence of PP. PP was induced in a normal subject by inspiration through a fixed resistance while mouth pressure was monitored.

FIG. 4B depicts representative PP data from a blood pressure monitor (FINAPRES) and oximetry plethysmograph recorded simultaneously using a correlation of variable degrees of induced PP, measured by a blood pressure monitor with changes in the plethysmographic waveforms from an oximeter. The transfer function relating voltage to mmHg can be inferred from the line drawn.

FIG. 5 depicts a schematic of a device used to measure pulsus paradoxus. This device uses two forms of waveform analysis, e.g., period amplitude analysis and power spectrum analysis, of a plethysmographic waveform obtained by a cardio device, e.g., a pulse oximeter, and combines them so that a measurement of pulsus paradoxus and a reliability index is output.

FIG. 6 depicts a transfer function of amplitude of power spectrum of plethysmography to pulsus paradoxus, Y=0.018 x²−0.213x+0.647.

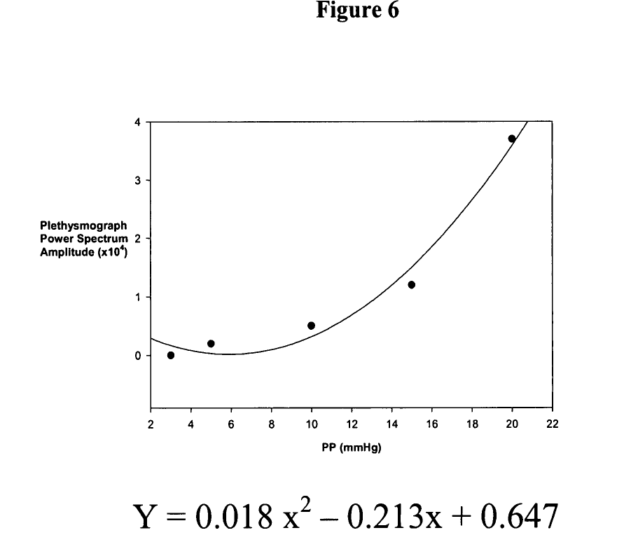

FIG. 7A depicts pericardial tamponade secondary to post-cardiotomy syndrome.

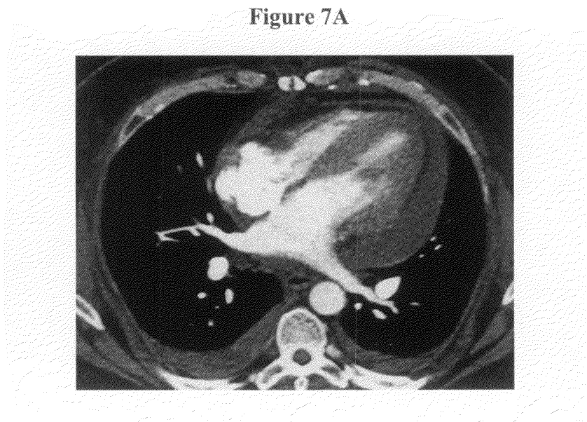

FIG. 7B depicts ECG and oximetry plethysmography of pericardial tamponade secondary to post-cardiotomy syndrome.

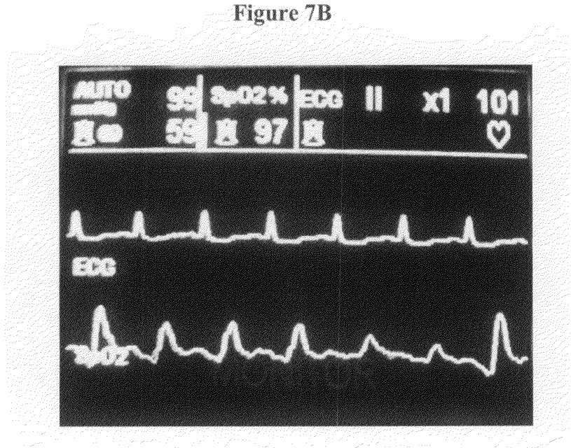

FIG. 8 depicts the Status Asthmaticus Continuum, the relationship between severity of respiratory distress to pulsus paradoxus and SpO2 (percentage oxygenated hemoglobin) as observed in the presence of symptoms or conditions such as tachycardia and tachypnea, ability to only speak a few words, hypoxia, “silent chest”, mixed metabolic acidosis and respiratory alkalosis, and metabolic acidosis pH<7.2, cardiac dysfunction, hypotension.

FIG. 9 depicts the power spectra of six different plethysmographic waveforms obtained under varying degrees of induced respiratory distress to cause pulsus paradoxus, including the baseline, 5 mmHg, 10 mmHg, 15 mmHg, 20 mmHg, and 25 mmHg. The amplitude of a waveform component having the frequency associated with respiration is indicated by an arrow; this “respiration” amplitude steadily increases with the severity of the induced respiratory distress.

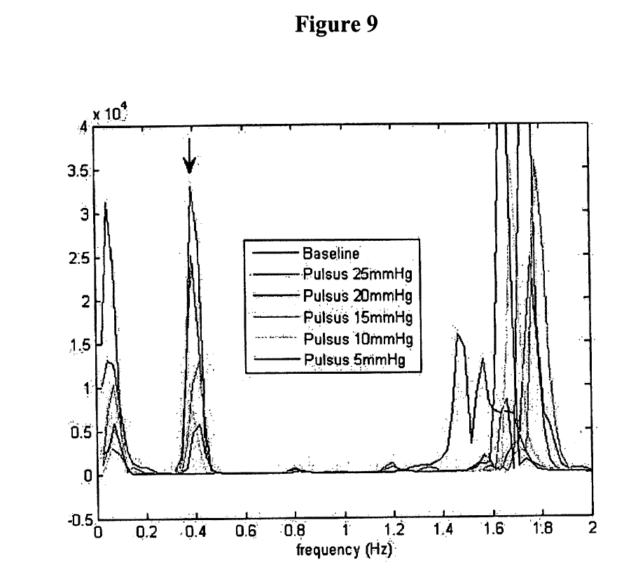

FIG. 10 depicts the power spectra of five different blood pressure waveforms (in mmHg) obtained under varying degrees of induced respiratory distress to cause pulsus paradoxus, including the baseline, 5 mmHg, 10 mmHg, 15 mmHg, and 20 mmHg from 0 to 10 Hertz (top) and 0 to 1 Hertz (bottom). The amplitude of a waveform component having the frequency associated with respiration is indicated by an arrow in top and bottom graphs; this “respiration” amplitude steadily increases with the severity of the induced respiratory distress.

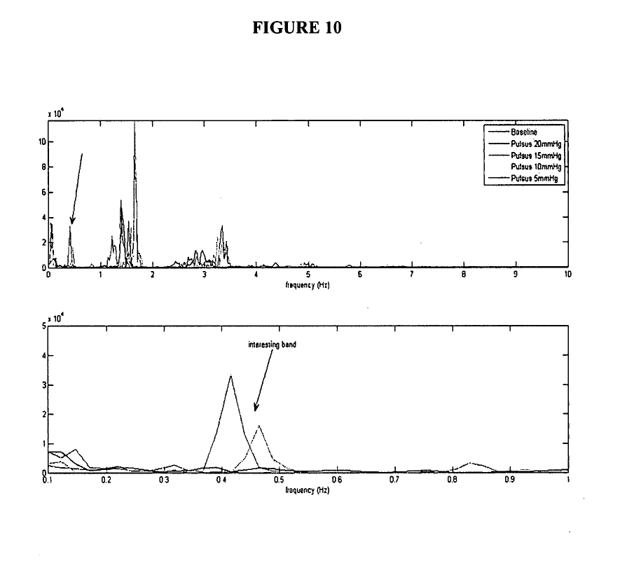

FIG. 11 depicts the power spectra of five different blood plethysmographic waveforms (in mmHg) obtained under varying degrees of induced respiratory distress to cause pulsus paradoxus, including the baseline, 5 mmHg, 10 mmHg, 15 mmHg, and 20 mmHg from 0 to 10 Hertz (top) and 0 to 1 Hertz (bottom). The amplitude of a waveform component having the frequency associated with respiration is indicated by an arrow in top and bottom graphs; this “respiration” amplitude steadily increases with the severity of the induced respiratory distress.

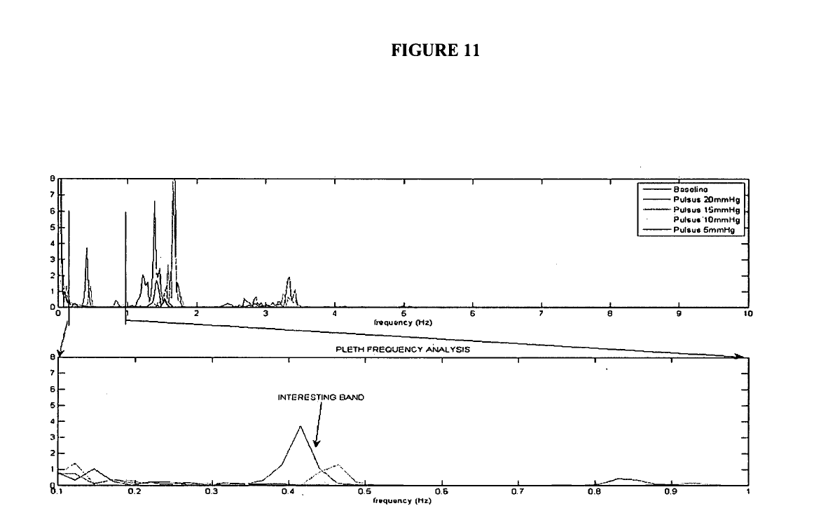

FIG. 12A depicts a plethysmographic waveform in volts measured under zero negative inspiratory pressure, our baseline (yielding a pulsus paradoxus of ˜2-3 mmHg).

FIG. 12B depicts a blood pressure waveform in mmHg measured under zero negative inspiratory pressure, our baseline (yielding a pulsus paradoxus of ˜2-3 mmHg).

FIG. 12C depicts a plethysmographic waveform in volts measured under negative 5 mmHg inspiratory pressure, our baseline (yielding a pulsus paradoxus of 5 mmHg).

FIG. 12D depicts a blood pressure waveform in mmHg measured under negative mmHg inspiratory pressure, our baseline (yielding a pulsus paradoxus of 5 mmHg).

FIG. 12E depicts a plethysmographic waveform in volts measured under negative 10 mmHg inspiratory pressure, our baseline (yielding a pulsus paradoxus of 13.7 mm Hg).

FIG. 12F depicts a blood pressure waveform in mmHg measured under negative mmHg inspiratory pressure, our baseline (yielding a pulsus paradoxus of 13.7 mmHg).

FIG. 12G depicts a plethysmographic waveform in volts measured under negative 15 mmHg inspiratory pressure, our baseline (yielding a pulsus paradoxus of 16.2 mmHg).

FIG. 12H depicts a blood pressure waveform in mmHg measured under negative mmHg inspiratory pressure, our baseline (yielding a pulsus paradoxus of 16.2 mmHg).

FIG. 12I depicts a plethysmographic waveform in volts measured under negative 20 mmHg inspiratory pressure, our baseline (yielding a pulsus paradoxus of 19.1 mmHg).

FIG. 12J depicts a blood pressure waveform in mmHg measured under negative mmHg inspiratory pressure, our baseline (yielding a pulsus paradoxus of 19.1 mmHg).

FIG. 13 depicts a hypothetical plethysmographic waveform generated by the function: f(x)=0.4 sin(x)+sin(6x)+1.5.

FIG. 14 depicts a schematic of a device used to measure respiratory distress by combining percentage oxygenated hemoglobin (SpO₂) and pulsus paradoxus as measured by a pulse oximeter.

### DETAILED DESCRIPTION

The invention features methods and devices for measuring pulsus paradoxus by combining various forms of analysis applied to pulsatile cardiorespiratory data. Waveform analysis can be used to diagnose respiratory distress in a subject. The waveforms associated with pulsatile cardiorespiratory data can be obtained using any of a number of devices currently utilized in a hospital setting. In addition to devices for obtaining pulsatile cardiorespiratory data, physician objective scoring of respiratory distress can be used. In addition to waveform analysis, other methods may be used to analyze cardiorespiratory data. Combinations of all or some of the methods can then be used to diagnose conditions in subjects, such as respiratory distress, by identifying pulsus paradoxus.

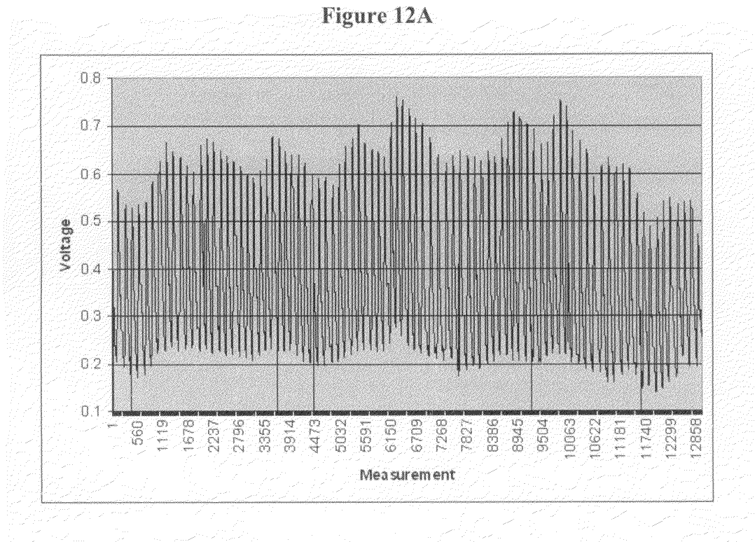

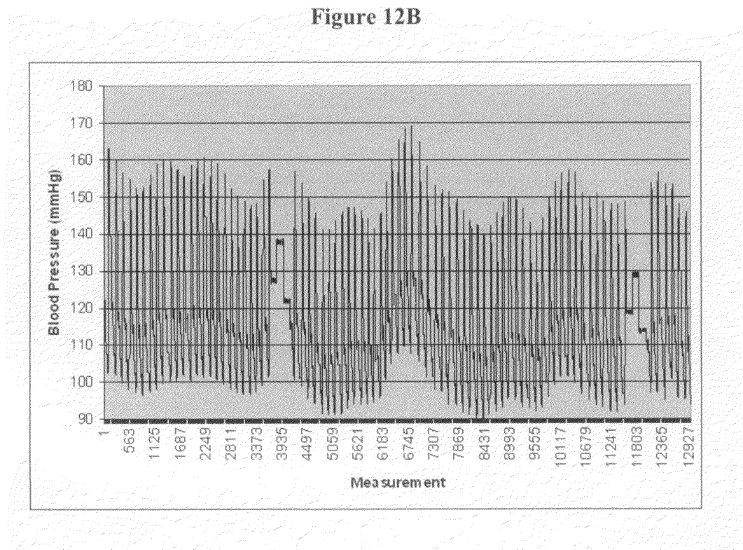

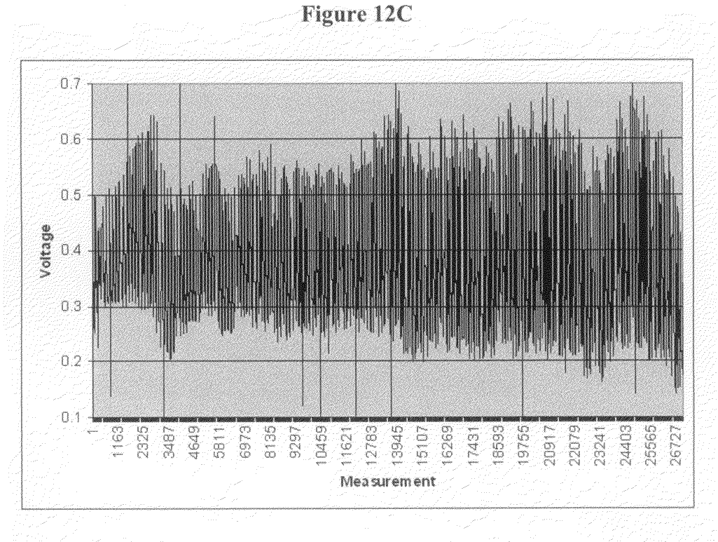

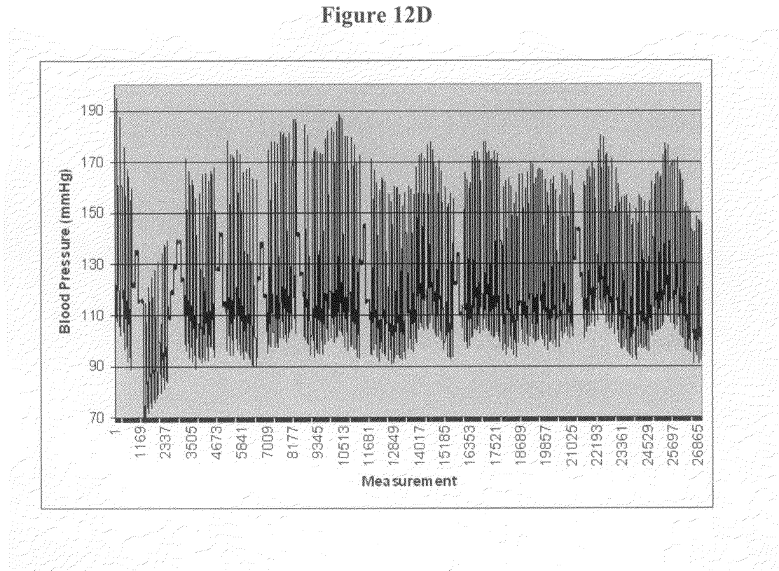

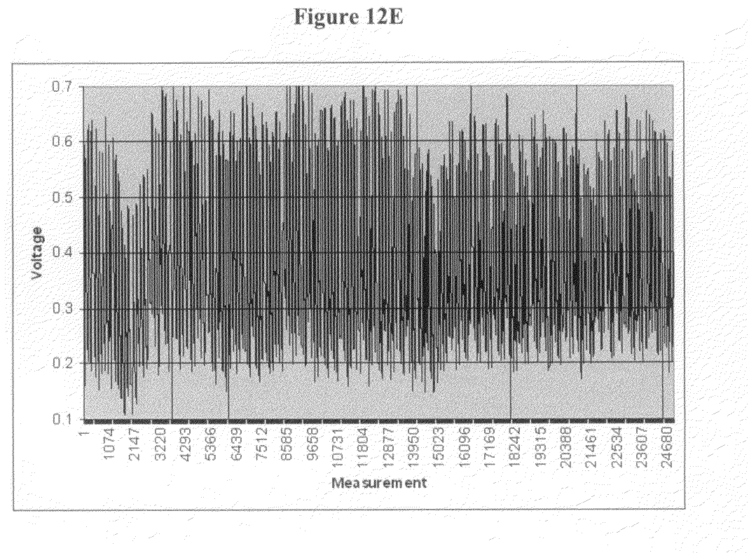

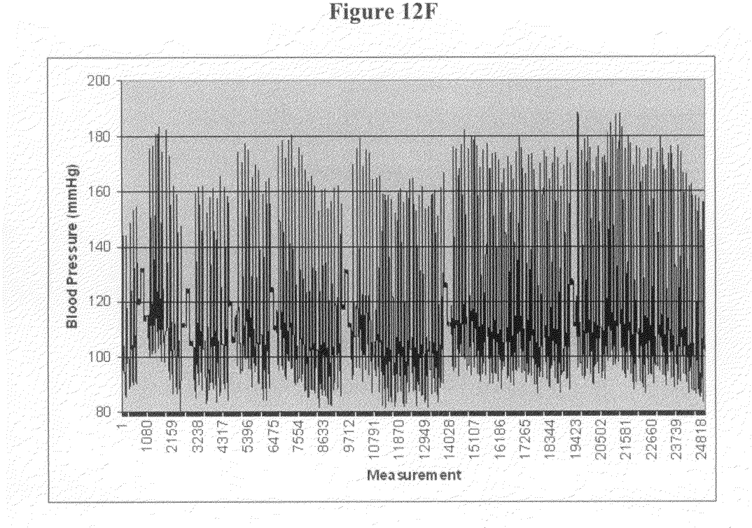

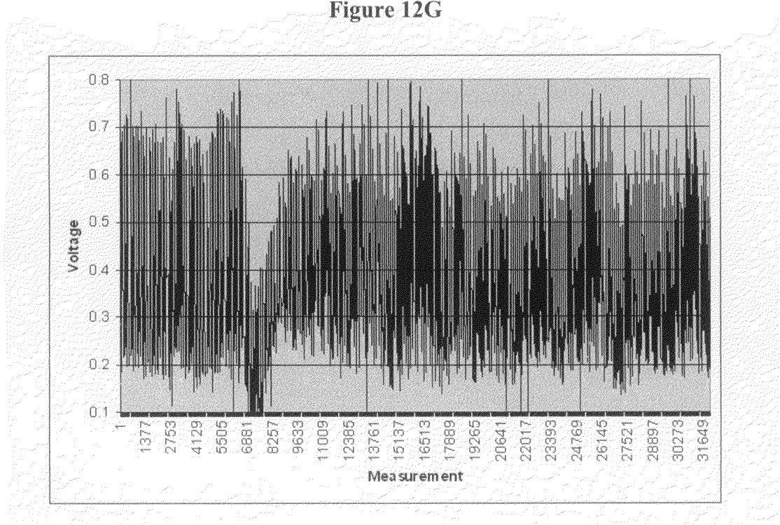

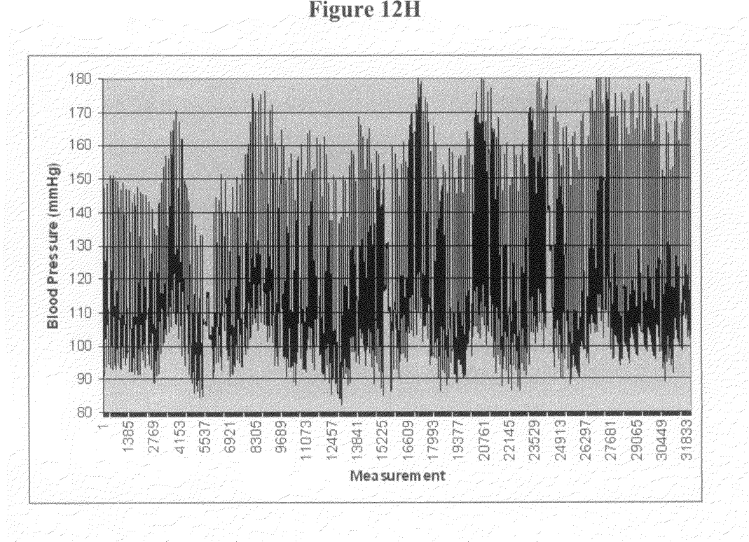

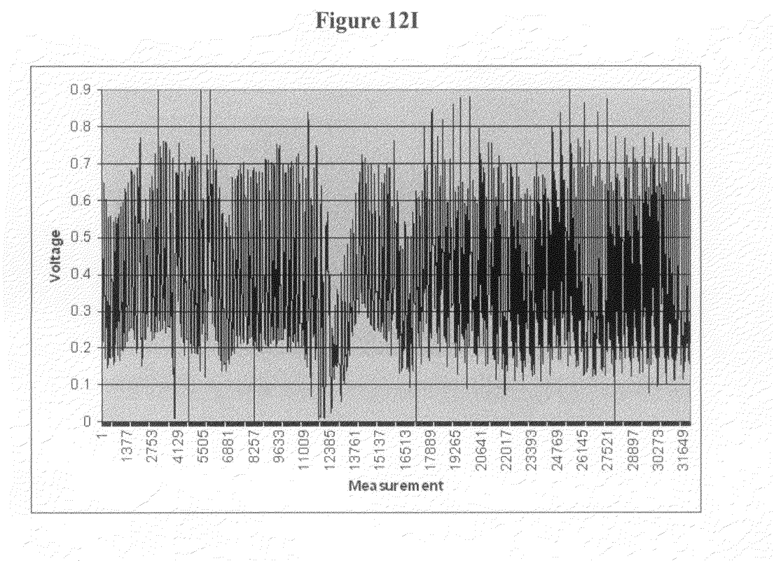

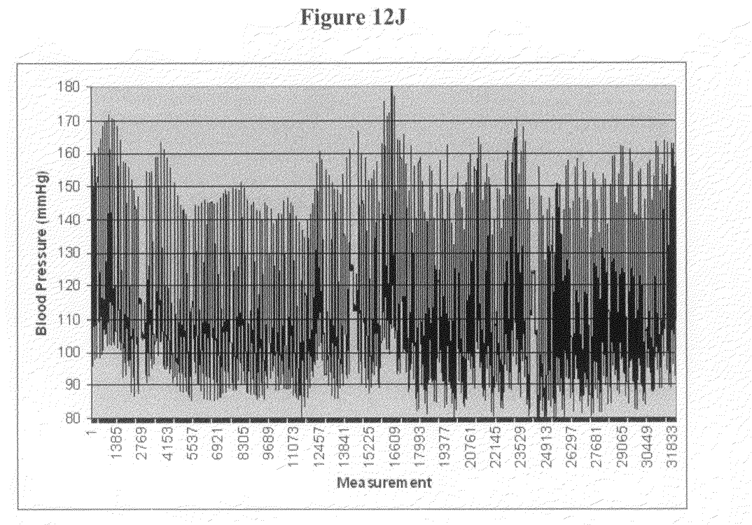

Various devices known in the art may be used to collect pulsatile cardiorespiratory data including, e.g., pulse oximeters, arterial tonometers, and finometers. These devices can be used to obtain plethysmographic waveforms, such as the ones shown in FIGS. 12A-12J, which were collected using the T-LINE (e.g., TL-150), or the PORTAPRES. The PRIMO™ handheld spot blood pressure monitoring device (Medwave, St. Paul, Minn.) can also be used to obtain plethysmographic waveforms. Pulsatile cardiorespiratory data may also be collected by physicians using, e.g., physician objective scoring of respiratory distress.

The combined waveform analysis outputs are then used to provide a measurement of pulsus paradoxus and, if present, to diagnose the presence or absence of respiratory distress.

The devices of the invention detect pulsus paradoxus by combining data collection devices such as pulse oximeters, arterial tonometers, or finometers with data compilation devices, such as computers that perform the mathematical techniques of the present invention, such that the final output of the method, a measurement of pulsus paradoxus and/or a diagnosis of respiratory distress, is displayed to a user on an output device.

Related methods of the invention include using a pulse oximeter to measure both pulsus paradoxus and the percentage of hemoglobin (Hb) that is saturated with oxygen (SpO₂) in a subject, wherein the percentage of O₂-saturated Hb is associated with a measure of the severity of respiratory distress and combined with the measurement of pulsus paradoxus which is also associated with a measure of the severity of respiratory distress, such that a diagnosis of respiratory distress or a recommendation of admission to hospital can be made. The measurement of the percentage of hemoglobin (Hb) which is saturated with oxygen may be associated with a rating of respiratory distress or a probability that a subject requires admission to a hospital, and the measurement of pulsus paradoxus may also be associated with a rating of respiratory distress or a probability that a subject requires admission to a hospital. These ratings or probabilities are then combined using any of the methods of combining, which are discussed below, e.g., by taking the maximum of the ratings or probabilities, to make a diagnosis of respiratory distress or a recommendation of admission to a hospital. Alternatively, the original measurements of pulsus paradoxus and O₂-saturated Hb, together, may be associated with a diagnosis of respiratory distress or a recommendation of admission to a hospital.

Other related methods of the invention include using a device of the invention to measure pulsus paradoxus in a subject, e.g., a device including a pulse oximeter, an arterial tonometer, or a finometer, and a physician's assessment, e.g., Physician Objective Scoring of Respiratory Distress, such that a diagnosis of respiratory distress or a recommendation of admission to hospital can be made. The measurement of pulsus paradoxus may be associated with a rating of respiratory distress or a probability that a subject requires admission to a hospital and the physician's assessment may also be associated with a rating of respiratory distress or a probability that a subject requires admission to a hospital. These ratings or probabilities are then combined using any of the methods of combining, which are discussed below, e.g., taking the maximum of the ratings or probabilities, to make a diagnosis of respiratory distress or a recommendation of admission to a hospital.

### Methods and Devices for Collecting Pulsatile Cardiorespiratory Data

### Physician Objective Scoring of Respiratory Distress

Physicians assessed each subject using eight visual analog scales (VAS) measuring: accessory muscle use, wheezing, prolonged expiratory phase, objective dyspnea, air entry, cyanosis, stemocleidomastoid muscle use, and mental status. Each scale ranged from 0 to 3, with anchor points at each integer. All of the scales were on the same side of a single sheet of paper. The physicians completed this assessment sequentially and filled in the form separately. They were instructed to mark the VAS scale with an “X” along the continuum which best reflected the subjects' conditions for each of the above physical exam findings. Scoring of these data was accomplished with a ruler, measuring the distance of the “X” from the origin for each scale.

### Measurement of Pulsus Paradoxus by Arterial Tonometer

Continuous blood pressure measurements were obtained non-invasively, for example, with a wrist mounted NCAT arterial tonometer (Nellcor, Pleasanton, Calif.). The analog output of this device was digitized, for example, via an 8-bit DAQ-500 analog to digital converter (National Instruments, Austin, Tex.). The sampling rate was 200 Hz.

### Measurement of Pulsus Paradoxus by a FINAPRES device

Continuous blood pressure was recorded non-invasively by a FINAPRES device (Ohmeda, Madison, Wis.). This device approximates invasive arterial blood pressure monitoring as well as the NCAT and has been used previously by our group and others. Data from the FINAPRES was digitized, for example, by a MP-100 analog-to-digital converter (Biopac Systems; Santa Barbara, Calif.), which created a text file that could be analyzed by the above pulsus paradoxus monitoring algorithm.

### Measurement of Pulsus Paradoxus by a Pulse Oximeter

Pulse plethysmography was obtained from a Nellcor 395 pulse oximeter (Pleasanton, Calif.) specially configured to separately record plethysmograph signals from the visible red and infrared photodiodes. Data transfer from the oximeter was accomplished digitally in real time through its analog signal output. Suitable oximeters include, for example, Biox 3700 and 3740 (Ohmeda Inc., Madison, Wis.), N-100 (Nellcor, Inc., Pleasanton, Calif.), and N-200 (Nellcor, Inc., Pleasanton, Calif.). The waveform is digitized by a suitable analog-to-digital converter, for example, an AD7861 available from Analog Devices located in Norwood, Mass.

### Measurement of Pulsus Paradoxus by Other Devices

Plethysmographic waveform data from a subject can be obtained by an optical plethysmograph and similarly coupled to analog-to-digital converters. Suitable plethysmographs include, for example, TSD 100B Optical Plethysmograph (BioPac Systems, Inc., Santa Barbara, Calif.). One can also utilize the T-LINE (e.g., TL-150), the PORTAPRES, or the PRIMO™ handheld spot blood pressure monitoring device (Medwave, St. Paul, Minn.). Waveforms, e.g., plethysmographic waveforms, may or may not have units of measurement, such as, e.g., mmHg or volts. Plethysmographic waveforms may include, without limitation, blood pressure waveforms and voltage waveforms collected by various devices, such as, e.g., pulse oximeters. Plethysmographic waveforms or pulsatile cardiorespiratory data may be collected with one or more devices at the same time or at different times on the same subject. If two or more devices are used to collect pulsatile cardiorespiratory data, then in a preferred embodiment of the invention, a transfer function relating blood pressure measured by one device to voltage changes observed in another device may be derived simultaneous to the collection of pulsatile cardiorespiratory data from one of the devices from which pulsus paradoxus will be determined (a typical transfer function will be a ratio relating mmHg to volts).

### Test Subjects

### Asthma Patients

Adult subjects 18-50 years of age with a documented history of asthma presenting with shortness of breath and probable asthma exacerbation were approached for study enrollment by trained clinical research assistants. Informed consent was obtained during the emergency department triage process or shortly thereafter, before emergency department treatment was initiated. Following subject consent, emergency department treatment was standardized and completed within 60 minutes according to NAEPP Guidelines: 3 sequential nebulized albuterol treatments and either intravenous Solumedrol 125 mg or oral Prednisone 60 mg. Just prior to the initiation and at the end of emergency department treatment, subjects' pulsus paradoxus by arterial tonometer was measured and both the treating physician and another physician performed objective asthma scoring. Physicians were blinded to AT-PP. Research assistants also measured subject vital signs during the AT-PP measurements. Following treatment, subject disposition was determined by the treating emergency physician blinded to AT-PP measurements. A poor outcome was defined as either subject admission or relapse of a discharged subject within 72 hrs. All discharged subjects were contacted to determine if they had an unscheduled visit for their asthma exacerbation after emergency department discharge. This study was reviewed and approved by the Institutional Review Board.

Medical records of enrolled subjects were analyzed to confirm that a prior diagnosis of asthma existed. Among admitted subjects, a physician blinded to AT-PP and the emergency department record, audited all inpatient records. Inappropriately admitted subjects were identified as those whose level of care could have been accomplished as an outsubject. These subjects were treated with oral steroids and metered dose inhalers and were not aggressively monitored.

### Induced Pulsus Paradoxus in Healthy Volunteer

Pulsus paradoxus was induced in a healthy adult using an established technique which involved having the subject breathe through a fixed resistance connected to a two-way nonrebreathing valve (Hans Rudolph; Kansas City, Mo.) attached to a manometer (OEM Medical; Marshalltown, Iowa). Airflow resistance occurred during inspiration, whereas expiration was unimpeded. The reference subject's blood pressure and oximetry plethysmograph were recorded continuously in the sitting position while he sequentially generated inspiratory mouth pressures from −5 to −20 mmHg in 5 mmHg increments using various devices including an arterial tonometer and a FINAPRES device. The subject controlled the generated mouth pressures by observing manometer readings. The respiratory rate was 20 breaths per minute.

### Waveform Analysis

### Period Amplitude Analysis Measuring Pulsus Paradoxus

Period amplitude analysis can be employed to analyze plethysmographic waveform data of a subject to measure the subject's pulse, respiration, or pulsus paradoxus. A periodic amplitude analysis algorithm was designed, for example, within LabVIEW® (National Instruments), which would identify peaks in blood pressure, including the local maxima, within the plethysmographic waveform data. Beat to beat systolic blood pressure (SBP) was identified using the algorithm recursively. Finally, the algorithm was applied again to the beat-to-beat SBP data to determine the variation in SBP with respiration, i.e., pulsus paradoxus. The algorithm calculates pulsus paradoxus by keeping a moving average of the last five peak SBPs and an average of the last five trough SBPs. Pulsus paradoxus is then calculated by subtracting the average trough SBP from the average peak SBP. Since the algorithm is able to monitor the maxima and minima of SBP within a respiratory cycle, a derivation of respiratory rate can be performed by measuring the elapsed time for the five SBP's. Various other forms of period amplitude analysis can be used to analyze plethysmographic waveforms as well, e.g., determinations of the average difference in height of at least two peaks, the average difference in area under at least two peaks, the average difference in slope of at least two peaks, or the average difference in curve length of at least two peaks present in plethysmographic waveform data. There are also various other forms of period amplitude analysis that do not require averaging which can be used to analyze plethysmographic waveforms, e.g., determinations of the maximum difference in height of at least two peaks, the maximum difference in area under at least two peaks, the maximum difference in slope of at least two peaks, or the maximum difference in curve length of at least two peaks present in plethysmographic waveform data. Period amplitude analysis may also include taking the average of maximum differences in peak features, such as, e.g., height, area under the curve, slope, curve length, or taking the maximum difference of averaged peak features. Various forms of period amplitude analysis are known in the art. Once period amplitude analysis is used to measure peak differences, those differences can be converted to differences in blood pressure measured in mmHg associated with pulsus paradoxus using a transfer function, such as, e.g., 1 mmHg/0.01V.

Once collected, continuous blood pressure data from the study subject was analyzed to calculate pulsus paradoxus using a period amplitude analysis algorithm. Text files from the oximeter plethysmograph were analyzed, e.g., by MP100 software (Biopac Systems; Santa Barbara, Calif.). A change in inspiratory and expiratory plethysmographic pulse amplitude caused by pulsus paradoxus was calculated for at least 10 respirations in each induced pulsus paradoxus data file and mean ±SD was calculated.

Devices used to perform period amplitude analysis are described more fully in, e.g., U.S. Pat. No. 6,325,761 and U.S. Pat. No. 6,129,675, both of which are incorporated by reference.

### Power Spectrum Analysis Measuring Pulsus Paradoxus

Power spectrum analysis, also known as Fourier analysis or Fourier transformation, and its variants, such as Fast Fourier Transformation, are used to identify the composition of waveforms. As applied to plethysmographic waveform data, power spectrum analysis can decompose a plethysmographic waveform into its composite signals so that the amplitude and frequency associated with the pulse of a subject and those associated with respiration and pulsus paradoxus can be separately identified.

A waveform, such as a plethysmographic waveform, represented by the function f(x) with a period of L can be decomposed into sine and cosine functions:

$$f ( x ) = {\sum }_{k = - ∞}^{∞} {A}_{k} {e}^{i ( 2 π kx / L )}$$

$${A}_{k} = \frac{1}{L} {\int }_{- L η}^{L η} f ( x ) {e}^{- i ( 2 π kx / L )} d x .$$

where e^{-i(2kx/L)}=cos(2Πkx/L)−i sin(2Πkx/L)

The coefficients Aₙ are the amplitudes associated with the waveform's composite signals represented by their respective sine and cosine functions. Using a Fast Fourier Transformation (also called a Discrete Fourier Transformation), these coefficients can also be calculated quickly by approximating the integrals with summations. Given a series of points {x[n]} along f(x), the series x[n] can be represented:

$$x [ n ] = \frac{1}{N} {\sum }_{k = 0}^{N - 1} X [ k ] {e}^{i2π \frac{k}{N} n}$$

$$n = 0 , 1 , … , N - 1$$

where the coefficients X[k] are calculated:

$$X [ k ] = {\sum }_{n = 0}^{N - 1} x [ n ] {e}^{- i2π \frac{k}{N} n}$$

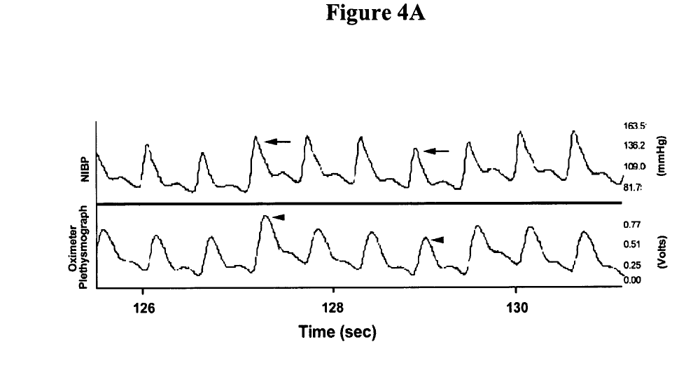

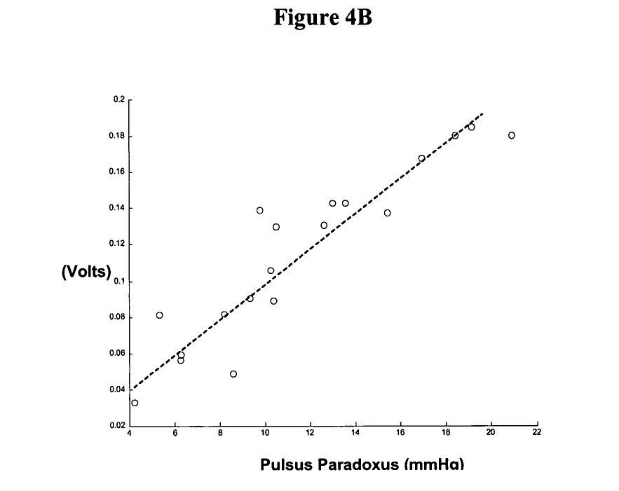

Once the waveform is decomposed, the signal associated with respiration and the signal associated with pulse can be identified. Each signal has its own characteristic period and amplitude, and the amplitude of the signal associated with respiration, either a coefficient Aₖ or a coefficient X[k], is then obtained. This amplitude associated with respiration indicates the amount of pulsus paradoxus, where larger amounts of pulsus paradoxus, such as those caused by respiratory distress, are associated with larger signal amplitudes. This amplitude can then be converted to a difference in blood pressure measured in mmHg associated with pulsus paradoxus using a transfer function, such as, e.g., 0.01 V/1 mmHg (FIG. 4B).

Devices used to perform power spectrum analysis are described more fully in U.S. Pat. No. 6,325,761 and U.S. Pat. No. 6,129,675, both of which are incorporated by reference.

### Matrix Singular Value Decomposition Analysis Measuring Pulsus Paradoxus

Singular value decompositions (SVD) can be employed to analyze plethysmographic waveform data of a subject to measure the subject's pulse, respiration, or pulsus paradoxus. SVD has advantages over methods such as power spectrum analysis because it can model periodicity using functions other than sine and cosine functions. A plethysmographic waveform is a time-series of measurements of blood pressure that can be evenly divided into time segments. Each time segment has the same number of points and each of those segments occupies a row of a matrix Aₙ. If the waveform is perfectly periodic and the time segment is equal to the length (or a multiple of a length) of the period, then the rows of the matrix are identical and, upon singular value decomposition, only one non-zero singular value s₁ and one periodic pattern v₁ (column vector) in the SVD matrices will be present. In practice, plethysmographic waveform data is rarely perfectly periodic, so perturbations (such as pulsus paradoxus) occurring over the course of multiple periods (such as multiple heart beats) will have additional non-zero singular values and period patterns. Any length can be chosen for the time segments, so an optimal time segment length is empirically selected such that the ratio of the first singular value s₁ relative to the second singular value s₂ is maximized. Upon selection of period length n, the matrix Aₙ is decomposed into the SVD matrices UΣV:

$${A}_{n} = \begin{matrix}x ( 1 ) & x ( 2 ) & ⋯ & x ( n ) \\ x ( n + 1 ) & x ( n + 2 ) & ⋯ & x ( 2 n ) \\ . & . & . & . \\ . & . & . & . \\ . & . & . & . \\ x ( nm - n + 1 ) & x ( nm - n + 2 ) & ⋯ & x ( nm )\end{matrix}$$

Aₙ=UΣV^{T}, where U and V are orthogonal matrices,
UU^{T}=U^{T}U=I, S=diag(s₁, s₂, . . . , sᵣ), r=min(m,n), and sᵢ≧sᵢ₊₁

The magnitude of the singular value sᵢ measures the contribution of the periodic pattern vector vᵢ. In the event of pulsus paradoxus, a greater second singular value s₂ (and possibly the higher order singular values) indicates a greater contribution of respiration to blood pressure, i.e. a greater pulsus paradoxus.

Linear algebraic methods of analyzing plethysmographic waveform data, such as singular value decomposition, covariance matrices, nonlinear analysis, or calculation of correlation dimension, can be used to determine the presence of pulsatile perturbations, such as pulsus paradoxus, and are described in more detail in, e.g., Bhattacharya et al. (IEEE Transactions on Biomedical Engineering 48:5-11, 2001) which is incorporated by reference.

### Methods of Combining

### Averages, Sums, Products, and Extrema

Two or more measurements, such as, e.g., measurements of pulsus paradoxus or a probability of admission based on a pulsus paradoxus measurement, can be combined by averaging them, adding them, multiplying them, or taking the maximum or minimum among them. Various forms of averaging include the median, mean, or mode. To yield a sum, measurements are typically added arithmetically. It is also possible to multiply two measurements or to add the reciprocals of two measurements to obtain a product or sum, preferably depending on whether or not those measurements are log-transformed or log-transformable. The extrema of a number of measurements typically include the maximum or minimum values of a distribution. The maximum and minimum values may be local or global. In preferred embodiments, the maximums and minimums may be selected from the peak heights observed within a distribution or a waveform, such as a plethysmographic waveform. In period amplitude analysis of a plethysmographic waveform obtained by pulse oximetry, the maximum peak height is compared to the minimum peak height along a waveform, in which the minimum peak height is probably not a global minimum considering that the troughs of the waveform surround the peaks, and by definition, are not included in the analysis of “peak height”. The average, sum, product, or extrema of other peak features may also be selected, e.g., the maximum area under a peak, the maximum slope of a peak, and maximum curve length of a peak, may be selected. Likewise, the average, sum, product, or extrema of the differences in peak features may be identified, as in period amplitude analysis of a plethysmographic waveform obtained by pulse oximetry, e.g., the maximum difference in area under any two peaks of a waveform, the maximum difference in height of any two peaks of a waveform, the maximum difference in slope of any two peaks of a waveform, and the maximum difference in curve length of any two peaks of a waveform.

### Kappa Statistic

Kappa statistic as used herein refers to any one of several similar measures of agreement among two or more ratings used with categorical data, e.g., Cohen's Kappa or the Weighted Kappa. Cohen's Kappa is used to compare only two raters, whereas other versions of the Kappa statistic compare more than two raters. Typically, the Kappa statistic measures the degree to which two or more sets of ratings of the same data agree in assigning the data to categories, for example, measuring the agreement of independent subject assignments to a category of subjects requiring medical admission or a category of subjects not requiring medical admission, based on ratings of subject pulsus paradoxus data. In the preferred embodiment of this invention, pulsus paradoxus as measured using period amplitude analysis of a plethysmographic waveform obtained by pulse oximetry is compared to pulsus paradoxus as measured using power spectrum analysis of the same waveform and the degree to which the pulsus paradoxus measurements agree in identifying patient's in need of hospital admission is measured by the Kappa statistic. If each of M subjects is assigned to one of n categories, e.g., one category of patients requiring hospital admission and another category not requiring admission, by k raters, e.g., ratings by period amplitude analysis and ratings by power spectrum analysis, then the Kappa statistic (K) is the ratio P(Actual)−P(Expected)/1−P(Expected), where P(Actual) is the fraction of the times the k raters agree and P(Expected) is the fraction of times the k raters are expected to agree by chance alone. In the preferred embodiment of this invention, P(Actual) is the fraction of times period amplitude analysis and power spectrum analysis agree in their recommendations to admit subjects and P(Expected) is typically 0.50. Perfect agreement corresponds to K=1, lack of agreement corresponds to K=0, and perfect disagreement yields a negative number. Ratings may be performed on data obtained, e.g., from different subjects or data obtained under different conditions or at different times from the same subject. Usually, but not necessarily, more than one piece of data is rated, such as, e.g., the ratings of 63 patients requiring admission to a hospital or not, discussed in the examples section.

### Correlation Coefficient

Correlation coefficients may be calculated to relate the degree to which measurements of pulsus paradoxus using one form of waveform analysis, e.g., period amplitude analysis, agree with measurements of pulsus paradoxus using another form of waveform analysis, e.g., power spectrum analysis. The measurements of pulsus paradoxus may be performed, e.g., on different subjects or under different conditions or at different times on the same subject.

### 50% Difference Standard

The agreement of two or more measurements, such as, e.g., measurements of pulsus paradoxus or a probability of admission based on a pulsus paradoxus measurement, can be assessed by determining whether or not the smallest such measurement is at least a fixed percentage, e.g., 50%, of the largest such measurement. Other fixed percentages may include, e.g., 10%, 20%, 30%, 40%, 50%, 60%, 70%, 80%, or 90%. In the preferred embodiments of the invention, a measure of pulsus paradoxus as determined using period amplitude analysis of a plethysmographic waveform obtained by pulse oximetry is compared to pulsus paradoxus as determined using power spectrum analysis of the same waveform, and a judgment that the measures of pulsus paradoxus agree is made when the smaller of the two measures is at least 50% of the larger of two values. Alternatively, two measurements may be said to agree, e.g., if they differ by an order of magnitude or a factor of 2, 3, 4, 5, 6, 7, 8, or 9.

### Averages, Sums, Products, and Extrema of P-values

P-values associated with measurements, just like the measurements themselves, can be combined, e.g., by averaging them, adding them, multiplying them, or taking the maximum or minimum among them. P-values describe the probability that a measurement equal to or greater than (or equal to or less than) a given measurement will belong to a given distribution. These p-values can be derived from empirical distributions or estimated using an estimated normal distribution and z-scores. In the preferred embodiment of the invention, a measurement of pulsus paradoxus can be associated with a p-value indicating the probability that measurement is derived from a healthy subject or a probability that measurement is derived from a subject experiencing respiratory distress, requiring admission to a hospital. If a given measurement of pulsus paradoxus is greater than the average measurement of a distribution of healthy subjects, the p-value describes the probability that a measurement equal to or greater than the given measurement belongs to a healthy subject. Likewise, if a given measurement of pulsus paradoxus is less than the average measurement of a distribution of subjects experiencing respiratory distress who require admission to a hospital, the p-value describes the probability that a measurement equal to or less than the given measurement belongs to a subject experiencing respiratory distress. P-values associated with measurements, such as, e.g., a measurement of pulsus paradoxus obtained by period amplitude analysis or power spectrum analysis of a plethysmographic waveform obtained by pulse oximetry, can aid in evaluating the significance of those measurements and making judgments, such as, e.g., whether or not to admit a subject to a hospital. Typically a threshold of significance is selected, e.g., 5%, 1%, 0.5%, 0.1%, such that p-values less than that threshold are considered significant and are used to make a judgment.

P-values can be assigned to more than one measurement, such as, e.g., a measurement of pulsus paradoxus by period amplitude analysis of a plethysmographic waveform obtained by pulse oximetry and a measurement of pulsus paradoxus by power spectrum analysis of the same waveform. The multiple p-values associated with multiple measurements of a common phenomenon, such as, e.g., pulsus paradoxus, may be combined by various means, e.g., by averaging them, adding them, multiplying them, or taking the maximum or minimum among them.

The various methods of combining p-values all involve choosing a means S(p₁, p₂, p₃, . . . ) for combining individual p-values p₁, p₂, p₃, . . . , constructing a combined p-value, and then optionally calculating the one-tailed probability of the combined p-value S(p₁, p₂, p₃, . . . ). Exemplary methods of combining p-values include:

1. The product of p₁, p₂, p₃, . . . (Fisher's rule);

2. The smallest of p₁, p₂, p₃, . . . (Tippett's rule);

3. The average of p₁, p₂, p₃, . . . ; and

4. The largest of p₁, p₂, p₃, . . .

The one-tailed probability of the combined p-value obtained using Fisher's rule can be obtained using the Chi-squared distribution. First, note that the cumulative distribution of a Chi-squared variate for two degrees of freedom is given by exp(−x/2). So, since p-values are by definition uniform between 0 and 1, −2·ln(p), where p is a p-value, is distributed as a Chi-squared with two degrees of freedom. In the next step, because Chi-squared variates are additive, the k Chi-squared variates with two degrees of freedom each when combined yield a Chi-squared variate with 2·k degrees of freedom. Therefore, to assess the significance (p-value of S) of the combined k p-values by Fisher's method, take twice the negative logarithm of their product, and compare it to the Chi-squared distribution for 2·k degrees of freedom, wherein the negative logarithm is deemed significant if it exceeds a critical Chi-value indicating significance, e.g., at the 5%.

For example, to combine two p-values p₁ and p₂, e.g., a p-value derived from using period amplitude analysis and a p-value derived using power spectrum analysis, you would calculate −2·ln(p₁·p₂) and assess its significance using Chi-squared distribution having four degrees of freedom. The density of such a Chi-squared distribution is x·exp(−x/2)/4, and the upper tail probability is (1+x/2)·exp(−x/2), where x=−2·ln(p₁·p₂). The general formula for upper tail probability of an arbitrary number of p-values is derived similarly: P·Σⱼ [−ln(P)]^{j}/j!, where P is the product of the n individual p-values, and the sum goes from 0 to n−1.

Other methods of combining p-values and assessing their significance include Mudholkar & George's t and Stouffer's overall Z. Using the p-values {pᵢ}, Mudholkar & George's t is calculated:

t=−sqrt((15k+12)/(5k+2)k²)Σ ln(pᵢ/(1−pᵢ))

The significance of Mudholkar & George's t is estimated using a t-distribution with 5k+4 degrees of freedom.

Alternatively, Stouffer's overall Z is calculated by first converting the p-values to z-scores. The overall z-score is then calculated:

Overall Z=Σ(Zᵢ)/sqrt(k),

Overall Z=Σ(wᵢ*Zᵢ)/Sqrt(Σ(w₁²)) (Liptak-Stouffer method), or

Overall Z=Σ(sqrt(wᵢ)*Zᵢ)/Sqrt(Σ(wᵢ))

Next the overall Z is then back transformed into an overall p-value using Rosenthal's Fail-safe N as a threshold by:

FSN=(ΣZᵢ/A)^{2-k},
where A=1.645 for α=0.05 and A=2.326 for α=0.01

Or, the overall Z is transformed into an overall p-value using Iyengar & Greenhouse's Worst case FSN as a threshold by:

FSNWC=[−B−sqrt(B²−4AC)]/2A,
where for α=0.05, A=0.01177, B=−0.217 ΣZᵢ−2.70554, and C=(ΣZᵢ)²2.70554 k, or
where for α=0.01, A=0.0007236, B=−0.0538 ΣZᵢ−5.4119, and C=(ΣZᵢ)²−5.4119 k

Once the final p-value for the combined p-values is obtained, it can be compared to a threshold of significance, e.g., α=0.05 or 0.01, and a judgment about the original measurements, e.g., pulsus paradoxus measurements, can be made, such as, e.g., that a subject is experiencing respiratory distress and requires admission to a hospital.

### Likelihood

Given different measurements, e.g., a measurement of pulsus paradoxus using period amplitude analysis and a measurement using power spectrum analysis, and a probability that each measurement will motivate a judgment, e.g., admission of a subject to a hospital, the probability of those measurements can be combined into a single likelihood score indicating the probability that the combination of those measurements would motivate that judgment. For each measurement Mᵢ with its associated probability P(Mᵢ) of motivating a judgment (assuming each judgment is independent), the combined likelihood L is:

L=ΠP(Mᵢ)

Likewise, the log-likelihood Log(L) is:

Log(L)=Σ log P(Mᵢ)

The assumption that the judgments are independent is the naïve Bayes assumption: it tends to work well in practice as known by those skilled in the art.

In an embodiment of the invention, the probability associated with a measurement of pulsus paradoxus obtained by period amplitude analysis of a subject's plethysmographic waveform and the probability obtained using power spectrum analysis can be combined into a single likelihood score by multiplying them, wherein that likelihood score is compared to a desired threshold to assess its significance and potentially used to make a diagnosis of respiratory distress or a recommendation that a subject be admitted to a hospital.

### Device for Measuring Pulsus Paradoxus

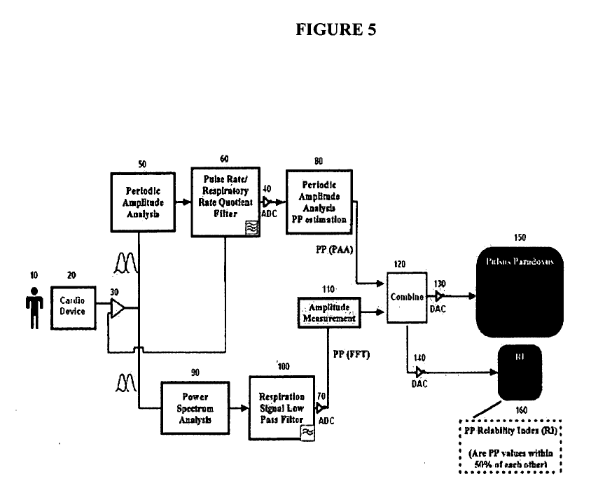

The devices of the invention perform and combine multiple forms of waveform analysis of pulsatile cardiorespiratory data to measure pulsus paradoxus. An exemplary device of the invention comprises one or more of components 10, 20, 30, 40, 50, 60, 70, 80, 90, 100, 110, 120, 130, 140, 150, and 160 connected as shown in FIG. 5. In an exemplary device of the invention, a cardio device (20) which can be, e.g., a pulse oximeter, an arterial tonometer, or a finometer, collects from subject (10) a pulsatile cardiorespiratory signal. Cardio device (20) is connected to differential amplifier (30), which accepts the pulsatile cardiorespiratory signal from cardio device (20) as input and a second signal associated with abnormal pulsatile cardiorespiratory data from filter (60) as a second input. Differential amplifier (30) takes the difference of the two input signals (i.e. the difference signal), effectively cancelling abnormal pulsatile cardiorespiratory data or allowing the passage of normal signals. Differential amplifier (30) sends the difference signal to both period amplitude analysis (PAA) device (50) and power spectrum analysis (PSA) device (90), e.g., a fast Fourier transform (FFT) processor. Exemplary PAA devices and PSA devices, to be used as components (50) and (90), are described in U.S. Pat. No. 6,325,761 and U.S. Pat. No. 6,129,675, both of which are incorporated by reference. The period amplitude analysis output (i.e. output signal) from PAA device (50), which includes a calculated pulse rate (i.e. a pulse frequency), a calculated respiratory rate (i.e. a respiratory frequency), and a calculated difference between waveform peaks, and the difference signal is then sent to filter (60), which either passes the calculated difference between waveform peaks if the pulse frequency is 3 to 7 times the respiration frequency or redirects the difference signal to differential amplifier (30) if the pulse frequency is less than 3 or greater than 7 times that of respiration. If the calculated difference between waveform peaks is passed by filter (60), it is sent to analog-to-digital (ADC) converter (40) where it is converted to a digital PAA signal and that digital PAA signal is then sent to digital processor (80) which estimates pulsus paradoxus using a transfer function or a look-up table stored on a chip or other computer readable medium that relates the calculated difference between waveform peaks of the first part of the signal in volts to a measurement of PP in mmHg.

PSA device (90) calculates and sends a signal encoding the power spectrum of the difference signal to low-pass filter (100), which isolates a signal component associated with respiration. The signal component associated with respiration from low-pass filter (100) is then sent to analog-to-digital (ADC) converter (70) which converts it to a digital respiration signal and that digital respiration signal is sent to digital processor (100), which measures the amplitude of the signal component associated with respiration and then estimates pulsus paradoxus using a transfer function or a look-up table stored on a chip or other computer readable medium that relates the amplitude of the signal component associated with respiration to a measurement of PP in mmHg.

The measurement of pulsus paradoxus in mmHg from digital processor (80) and the measurement of pulsus paradoxus in mmHg from digital processor (110) are both sent to digital processor (120), which combines the two measurements of pulsus paradoxus in mmHg (i.e., the two waveform analysis outputs), by, e.g., finding the average, sum, product, or extremum of a group of waveform analysis outputs, calculating a Kappa Statistic or correlation coefficient relating waveform analysis outputs, finding differences between waveform analysis outputs, finding the average, sum, products, and extremum of p-values associated with the waveform analysis outputs, or calculating the likelihood of the waveform analysis outputs, yielding a combined measurement of pulsus paradoxus and a reliability index (RI). The preferred reliability index (RI), e.g., is a correlation coefficient or a Kappa statistic relating the two measurements of pulsus paradoxus collected at multiple time points from subject (10) or a logical function that indicates whether or not the two measurements of pulsus paradoxus in mmHg differ by 50% or more, i.e., if the maximum of the two measurements of pulsus paradoxus in mmHg−(minus) the minimum of the two measurements of pulsus paradoxus in mmHg>(is greater than) the minimum of the two measurements of pulsus paradoxus in mmHg, then the two measurements of pulsus paradoxus are “reliable”, otherwise they are “unreliable”. The combined measurement of pulsus paradoxus from digital processor (120) is sent to digital-to-analog converter (DAC) (130) and displayed on output device (150), e.g., a monitor or an LED display. The combined reliability index (RI) is then sent to digital-to-analog converter (DAC) (140) and displayed on output device (160), e.g., a monitor, an LED display, or the output device (150).

In alternate embodiments of the invention, the device as depicted in FIG. 5 may have substituted PAA device (50) or PSA device (90) with a SVD device, e.g., a processor, which performs singular value decomposition, calculates a singular value associated with respiration, and outputs a signal that is eventually passed to a digital processor (80) or (10) which uses a transfer function or a look-up table stored on a chip or other computer readable medium that relates the calculated singular value associated with respiration to a measurement of pulsus paradoxus in mmHg.

In other embodiments of the invention, the device depicted in FIG. 5 may have one, two, three, four, or more digital processors that perform the functions of one or more components of (30), (50), (60), (80), (90), (100), (110), and (120), connected to the other components of the device as shown. For example, the digital processor(s) of a computer, which also includes software, memory buffers, RAM, and hard disk drives, may be used by the devices of the invention.

In an alternate embodiment of the invention, the device in FIG. 5 may be a cardio device (20) connected to a computer or a cardio device connected to an analog-to-digital converter connected to a computer. The computer of the device may include a digital processor, software, memory buffers, RAM, and hard disk drives and may further include the functionality of one or more of the components 30, 40, 50, 60, 70, 80, 90, 100, 110, 120, 130, 140, 150, and 160.

In other embodiments of the devices of the invention, a digital component may be substituted for an analog component having a similar function or an analog component may be substituted for a digital component having a similar function. The components of the devices of the invention may be coupled to analog-to-digital or digital-to-analog converters. Components of the devices of the invention may be substituted by other components that perform similar functions.

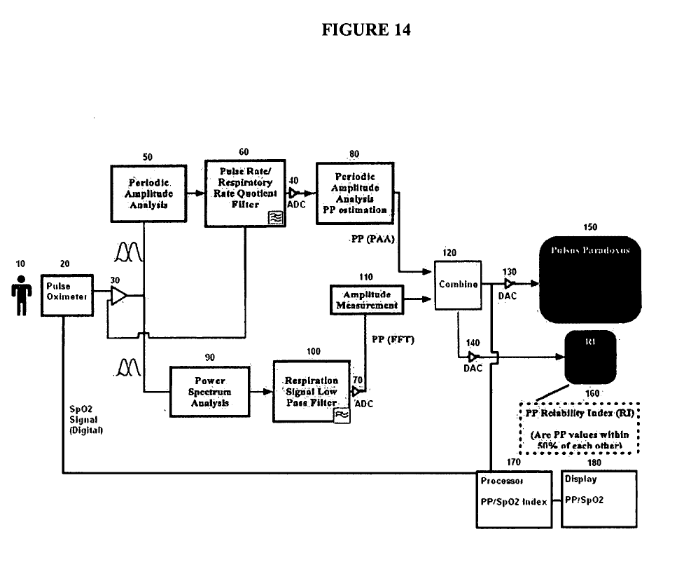

In other embodiments of the devices of the invention, digital processor (170) and output device (180) may be connected to the device in FIG. 5, as further depicted in FIG. 14. Pulse oximeter (20) generates a digital signal encoding the percentage oxygenated hemoglobin of a subject (SpO₂) which is sent to digital processor (170) which also receives a signal carrying a pulsus paradoxus measurement from digital processor (120). Digital processor (170) combines the SpO₂ and pulsus paradoxus measurement signals and generates a PP/SpO₂ index or some combined measurement of respiratory distress, which is sent to an output device (180), which may be coupled to a DAC. A device of the invention may also have any number of components shown in FIG. 14 that are omitted or substituted with components of equivalent function. An exemplary device of the invention comprises one or more of components 10, 20, 30, 40, 50, 60, 70, 80, 90, 100, 110, 120, 130, 140, 150, 160, 170, and 180 connected as shown in FIG. 14.

### Device Components

The devices of this invention may use any of the components described below or components that are functionally similar or equivalent.

Pulse Oximeters are simple non-invasive devices used to monitor the percentage of hemoglobin (Hb) which is saturated with oxygen. The pulse oximeter consists of a probe attached to the patient's finger or ear lobe which is linked to a computerized unit. The unit displays the percentage of Hb saturated with oxygen together with an audible signal for each pulse beat, a calculated heart rate and in some models, a graphical display of the blood flow past the probe. Exemplary pulse oximeters used in the devices of the invention include Ohmeda, Inc. Biox 3700 and 3740; Nelicor N-100 and N-200; Nonin Onyx 9500 Pulse Oximeter; SPO 5500 Finger Pulse Oximeter; Pulse Check Mate Blood Oxygen Saturation & Pulse Sport Finger Oximeter; BCI Autocorr Digital 3304 Pulse Oximeter; Sammons Preston Handheld Pulse Oximeter; Pulse Oximeter PalmSAT 2500 Handheld Pulse Oximeter; Nonin Avant 22 BP Monitor and Pulse Oximeter; SPO Medical PulseOx 5500 Finger Pulse Oximeter; and BCI 3303 Hand-held Pulse Oximeter.

Arterial tonometers are devices for blood-pressure measurement in which an array of pressure sensors is pressed against the skin over an artery. Exemplary arterial tonometers used in the devices of the invention include the NCAT arterial tonometer—Nellcor, Pleasanton, Calif.; the Colin radial artery tonometer; and the devices described in U.S. Pat. Nos. 5,158,091 and 6,290,650, which are incorporated by reference.

Finometers are non-invasive stationary blood measurement and beat to beat hemodynamic monitoring systems. Finometers capture the continuous blood pressure waveform and may compute beat to beat hemodynamic parameters including Cardiac Output (CO), Stroke Volume (SV), Total Peripheral Resistance (TPR), Pulse Rate Variability (PRV) and BaroReflex Sensitivity (BRS). Exemplary finometers include FINAPRES devices, such as the FINOMETER(PRO & MIDI, the PORTAPRES, and the Richmond Pharmacology Finometer.

Exemplary software used by the devices of this invention to perform various calculations, e.g., period amplitude analysis, power spectrum analysis, singular value decompositions, averages, sums, correlation coefficients, Kappa statistics, etc., include mathematical libraries such as, e.g., IMSL, Numerical Recipes, MATLAB, SPSS, and possibly interfaces to programs such as, e.g., Mathematica, Maple, Spotfire, and Microsoft Excel.

Differential amplifiers (including difference amplifiers) amplify the difference between two input signals (−) and (+). Differential amplifiers are also referred to as a differential-input single-ended output amplifiers. Differential amplifiers are precision voltage differential amplifiers, and form the central basis of more sophisticated instrumentation amplifier circuits. Exemplary differential amplifiers used in the devices of the invention include THS4502 Texas Instruments—High-Speed Fully-Differential Amplifiers; THS4502CD Texas Instruments—High-Speed Fully-Differential Amplifiers; THS4502CDGK Texas Instruments—High-Speed Fully-Differential Amplifiers; THS4502CDGKR Texas Instruments—High-Speed Fully-Differential Amplifiers; THS4502CDGN Texas Instruments—High-Speed Fully-Differential Amplifiers; THS4502CDGNR Texas Instruments—High-Speed Fully-Differential Amplifiers; THS4502CDR Texas Instruments—High-Speed Fully-Differential Amplifiers; THS45021D Texas Instruments—High-Speed Fully-Differential Amplifiers; THS45021DGK Texas Instruments—High-Speed Fully-Differential Amplifiers; THS45021DGKR Texas Instruments—High-Speed Fully-Differential Amplifiers; THS45021DGN Texas Instruments—High-Speed Fully-Differential Amplifiers; THS45021DGNR Texas Instruments—High-Speed Fully-Differential Amplifiers; THS45021DR Texas Instruments—High-Speed Fully-Differential Amplifiers; THS4503 Texas Instruments—High-Speed Fully-Differential Amplifiers; THS4503CD Texas Instruments—High-Speed Fully-Differential Amplifiers; THS4503CDGK Texas Instruments—High-Speed Fully-Differential Amplifiers; THS4503CDGKR Texas Instruments—High-Speed Fully-Differential Amplifiers; THS4503CDGN Texas Instruments—High-Speed Fully-Differential Amplifiers; THS4503CDGNR Texas Instruments—High-Speed Fully-Differential Amplifiers; THS4503CDR Texas Instruments—High-Speed Fully-Differential Amplifiers; THS45031D Texas Instruments—High-Speed Fully-Differential Amplifiers; THS45031DGK Texas Instruments—High-Speed Fully-Differential Amplifiers; THS45031DGKR Texas Instruments—High-Speed Fully-Differential Amplifiers; THS45031DGN Texas Instruments—High-Speed Fully-Differential Amplifiers; THS45031DGNR Texas Instruments—High-Speed Fully-Differential Amplifiers; THS45031DR Texas Instruments—High-Speed Fully-Differential Amplifiers; AD629 Analog Devices—High Common-Mode Voltage Difference Amplifier; INA117 Texas Instruments—High Common-Mode Voltage Difference Amplifier; INA132 Texas Instruments—Low Power, Single-Supply Difference Amplifier; INA133 Texas Instruments—High-Speed, Precision Difference Amplifiers; INA143 Texas Instruments—High-Speed, Precision, G=10 or G=0.1 Difference Amplifiers; INA145 Texas Instruments—Programmable Gain Difference Amplifier; INA146 Texas Instruments High-Voltage, Programmable Gain Difference Amplifier; INA148 Texas Instruments—+−200V Common-Mode Voltage Difference Amplifier; INA152 Texas Instruments—Single-Supply Difference Amplifier; INA154 Texas Instruments—High-Speed, Precision Difference Amplifier (G=1); INA157 Texas Instruments—High-Speed, Precision Difference Amplifier; and MIC7201 Micrel—GainBlock™ Difference Amplifier.

Analog-to-digital converters (ADC) accept an analog input, e.g., a voltage or a current, and convert it to a digital value that can be read by a microprocessor: Exemplary types of ADCs are flash, successive approximation, and sigma-delta. Exemplary analog to digital converters (ADC) used in the devices of the invention include an 8-bit DAQ-500 analog to digital converter National Instruments, Austin, Tex.; MP-100 analog-to-digital converter Biopac Systems, Santa Barbara, Calif.; AD7861 Analog Devices, Norwood, Mass.; LTC1408 Linear Technology—6 Channel, 14-Bit, 600 ksps Simultaneous Sampling ADC with Shutdown; LTC2208 Linear Technology—16-Bit, 130Msps ADC; LTC2202 Linear Technology—16-Bit, 10Msps ADC; LTC2255 Linear Technology—14-Bit, 125Msps Low Power 3V ADCs; LTC2242-12 Linear Technology—12-Bit, 250Msps ADC; LTC2285 Linear Technology—Dual 14-Bit, 125Msps Low Power 3V ADC; LTC2442 Linear Technology—24-Bit High Speed 4-Channel ΔΣ ADC with Integrated Amplifier; LTC2498 Linear Technology—24-Bit 8-/16-Channel ΔΣ ADC with Easy Drive Input Current Cancellation; LTC2496 Linear Technology—16-Bit 8-/16-Channel ΔΣ ADC with Easy Drive Input Current Cancellation; MCP3002 Device from Microchip Technology Inc.; MCP3201 Device from Microchip Technology Inc.; and MCP3301 Device from Microchip Technology Inc.

Digital-to-Analog converters (DAC) are devices for converting digital (usually binary) code to analog signals (current, voltage or electric charge). Exemplary types of digital to analog converters (DAC) used in the devices of the invention include Pulse Width Modulator DACs, Oversampling DACs, Binary Weighted DACs, R-2R Ladder DACs, Segmented DACs, and Hybrid DACs. Exemplary digital to analog converters (DAC) include AD5624 Analog Devices—2.7 V to 5.5 V, 450 μA, Rail-to-Rail Output, Quad, 12-/16-Bit nanoDACs®; AD5623R Analog Devices—Dual, 12-Bit nanoDAC® with 5 ppm/° C. On-Chip Reference; and AD5664R Analog Devices—Quad, 16-Bit nanoDAC® with 5 ppm/° C. On-Chip Reference.

Digital processors (microprocessors) are digital electronic component with transistors on a single semiconductor integrated circuit (IC). One or more microprocessors typically serve as a central processing unit (CPU) in a computer system or other device. Exemplary digital processors used in the devices of the invention include AMD K5, K6, K6-2, K6-III, Duron, Athlon, Athlon XP, Athlon MP, Athlon XP-M (Intel x86 architecture); AMD Athlon 64, Athlon 64 FX, Athlon 64×2, Opteron, Sempron, Turion 64 (AMD64 architecture); ARM family, StrongARM, Intel PXA2xx; Atmel AVR architecture (purely microcontrollers); EISC; RCA 1802 (a.k.a. RCA COSMAC, CDP1802); Cyrix M1, M2 (Intel x86 architecture); DEC Alpha; Intel 4004, 4040; Intel 8080, 8085, Zilog Z80; Intel 8086, 8088, 80186, 80188, 80286, 80386, 80486 (Intel x86 architecture); Intel Pentium, Pentium Pro, Celeron, Pentium II, Pentium III, Xeon, Pentium 4, Pentium M, Pentium D, Celeron M, Celeron D (Intel x86; parents of IA-64, with HP PA-RISC); Intel Itanium (IA-64 architecture); Intel i860, i960; MIPS architecture; Motorola 6800; MOS Technology 6502; Motorola 6809; Motorola 68000 family, ColdFire; Motorola 88000 (parents of the PowerPC family, with POWER); NexGen Nx586 (Intel x86 architecture); IBM POWER (parents of the PowerPC family, with 88000); NSC 320xx; OpenCores OpenRISC architecture; PA-RISC family (HP, parents to the IA-64 architecture, with x86); PowerPC family, G3, G4, G5; National Semiconductor SC/MP (“scamp”); Signetics 2650; SPARC, UltraSPARC, UltraSPARC II-IV; SuperH family; Transmeta Crusoe, Efficeon (VLIW architectures, Intel x86 emulator); INMOS Transputer; VIA's C3,C7,Eden Series (Intel x86 architecture); and Western Design Center 65xx. Exemplary fast Fourier transform (FFT) processors used in the devices of the invention include DASP/PAC—Honeywell; PDSP 16510A—Zarlink (Plessey,Mitel); PDSP16515A—Zarlink (Plessey,Mitel); L64280—LSI; Dassault—Electronique; TM-66—Texas Mem Sys; BDSP9124/9320—Butterfly DSP; Cobra—Colorado State; CNET—E. Bidet; Spiffee 1—Stanford; Spiffee Low Vt—Stanford; Spiffee ULP—Stanford; DaSP/PaC/RaS—Array Microsystems; SNC960A—Sicom; DSP-24—DSP Architectures; M. Wosnitza—ETH, Zurich; Radix—RDA108; DoubleBW; TM-44—Texas Mem Sys; S. M. Currie—Mayo FFT; PowerFFT—Eonic BV; and J.-C. Kuo—NTU. Microprocessors of the devices of the invention may be coupled to memory buffers, random access memory (RAM), or computer readable media, such as hard disk drives.

Electronic filters are electronic circuits which perform signal processing functions, specifically intended to remove unwanted signal components or enhance wanted ones. Electronic filters may be analog or digital. A digital filter is any electronic filter that works by performing digital mathematical operations on an intermediate form of a signal. Exemplary types of filters include bandpass filters, band-reject filters, Gaussian filters, Bessel filters, Butterworth filters, elliptical filters (Cauer filters), Linkwitz-Riley filters, Chebyshev filters, high-pass filters, and low-pass filters. Exemplary filters include HSP43124 Intersil—Filter, 24 Bit Serial I/O, 45 MHz, 256 Tap Programmable FIR Filter, 24-Bit Data, 32-Bit Coefficients; HSP43168 Intersil—Filter, Dual FIR, 33 MHz, Two Independent 8-TAP FIRs or a Single 16-TAP FIR, 10-Bit Data, 10-Bit Coefficients; HSP43216 Intersil—Filter, 52MSPS, 67-TAP Halfb& FIR with 20-Bit Coefficients, 16-Bit Inputs and Outputs; HSP43220 Intersil—Filter, Decimating Digital, 33 MHz, 16-Bit 2s Compliment Input, 24-Bit Extended Precision Output, 20-Bit Coefficients in FIR; HSP48901 Intersil—ImAge Filter, 3×3, 30 MHz, 1D and 2D Correlation/Convolution; Frequency Devices, Inc. 854 0.1 Hz to 102.4 kHz; Frequency Devices, Inc. 858 0.1 Hz to 102.4 kHz; Frequency Devices, Inc. D824 1 Hz to 102.4 kHz; Frequency Devices, Inc. D828 1 Hz to 102.4 kHz; Frequency Devices, Inc. 424 10 Hz to 102.4 Hz; Frequency Devices, Inc. 428 10 Hz to 102.4 Hz; Frequency Devices, Inc. 818 1 kHz to 1.28 MHz; Frequency Devices, Inc. D61 0.02 Hz to 1.0 Hz; Frequency Devices, Inc. DP64 1 Hz to 5 kHz; Frequency Devices, Inc. R854 1 Hz to 102.4 kHz; Frequency Devices, Inc. R858 1 Hz to 102.4 kHz; Frequency Devices, Inc. D824 1 Hz to 102.4 kHz; Frequency Devices, Inc. 824 1 Hz to 102.4 kHz; Frequency Devices, Inc. 828 1 Hz to 102.4 kHz; Frequency Devices, Inc. D64BP 1 Hz to 100 kHz; Frequency Devices, Inc. D68BP 1 Hz to 100 kHz; Frequency Devices, Inc. D100BP 100 Hz to 100 kHz; Frequency Devices, Inc. 824BP 1 Hz to 25.6 kHz; Frequency Devices, Inc. 828BP 1Hz to 25.6 kHz; Frequency Devices, Inc. D68BR 1 Hz to 100 kHz; and Frequency Devices, Inc. 828BR 1 Hz to 25.6 kHz.

Comparators are devices which compare two voltages or currents and switch their output to indicate which of the two is larger. More generally, comparators refer to devices that compare two items of data. Exemplary comparators of the devices of the invention include 54AC520 National Semiconductor—8-Bit Identity Comparator; 54AC521 National Semiconductor—8-Bit Identity Comparator; 54ACT520 National Semiconductor—8-Bit Identity Comparator; 54ACT521 National Semiconductor—8-Bit Identity Comparator; 54F521 National Semiconductor—8-Bit Identity Comparator; 54FCT521 National Semiconductor—8-Bit Identity Comparator; 54LS85 National Semiconductor—4-Bit Magnitude Comparator; CD4063BMS Intersil—Digital Comparator, 4-Bit Magnitude, Rad-Hard, CMOS, Logic; CD4585BMS Intersil—Digital Comparator, 4-Bit Magnitude, 3 Cascading Inputs for Expanding Comparator Function, Rad-Hard, CMOS, Logic; DM9324 National Semiconductor—5-Bit Comparator; HCTS85MS Intersil—Comparator, Digital, Magnitude, 4-Bit, TTL Inputs, Rad-Hard, High-Speed, CMOS, Logic; MC 100E166 ON Semiconductor—5V ECL 9-Bit Magnitude Comparator; MC10E1651 ON Semiconductor—5V, −5V ECL Dual ECL Output Comparator With Latch; MC10E1652 ON Semiconductor—5V ECL Dual ECL Output Comparator With Latch; MXL1016 Maxim—Ultra-Fast Precision TTL Comparator; and MXL1116 Maxim.

Buffers are a region of memory used to temporarily hold output or input data, which can be output to or input from devices outside the computer or processes within a computer. Buffers can be implemented in either hardware or software, but the vast majority of buffers are implemented in software. Exemplary buffers used by the devices of the invention include PDSP 16450 Plessey Digital Signal Processor; 100322 National Semiconductor—Low Power 9-Bit Buffer; 100352 National Semiconductor—Low Power 8-Bit Buffer with Cut-Off Drivers; 74ABT125 Philips Semiconductors—Quad buffer (3-State); 74ABT126 Philips Semiconductors—Quad buffer (3-State); 74AHC1G07 Philips Semiconductors—Buffer with open-drain output; 74VCX162400N Semiconductor—Low-Voltage 1.8/2.5/3.3V 16-Bit Buffer; 74VCX162440N Semiconductor—Low-Voltage 1.8/2.5/3.3V 16-Bit Buffer With 3.6 V-Tolerant Inputs and Outputs (3-State, Non-Inverting); 74VCXH16240 ON Semiconductor—Low-Voltage 1.8/2.5/3.3V 16-Bit Buffer; 74VCXH 16244 ON Semiconductor—Low-Voltage 1.8/2.5/3.3V 16-Bit Buffer; CD4010B Texas Instruments—CMOS Hex Non-Inverting Buffer/Converter; CD4041BMS Intersil—True/Complement, Buffer, Quad, Rad-Hard, CMOS, Logic; CD4041UBMS Intersil; CD4049UB Texas Instruments—CMOS Hex Inverting Buffer/Converter; CD4050B Texas Instruments—CMOS Hex Non-Inverting Buffer/Converter; CD4503BMS Intersil—Buffer, Hex, Tri-State, Rad-Hard, CMOS, Logic; CD4504BMS Intersil—Buffer, Voltage Level Shifter, TTL to CMOS, CMOS to CMOS, Hex, Rad-Hard, CMOS, Logic; and MC100E122 ON Semiconductor—5V ECL 9-Bit Buffer.

Fabrication, implementation, and applications of electronic devices are described in Sen M. Kuo, Woon-Seng Gan: Digital Signal Processors: Architectures, Implementations, and Applications, Prentice Hall; Stergios Stergiopoulos: Advanced Signal Processing Handbook: Theory and Implementation for Radar, Sonar, and Medical Imaging Real-Time Systems, CRC Press; P. Gaydecki: Foundations Of Digital Signal Processing: Theory, Algorithms And Hardware Design, Institution of Electrical Engineers; D. Bamaal: Analog Electronics for Scientific Application, Waveland Press, Inc.; and D. Bamaal: Digital and Microprocessor Electronics for Scientific Application, Waveland Press, Inc., each of which is incorporated by reference.

Any of the methods of combining, comparing, and mathematically evaluating measurements of pulsus paradoxus are compatible with any of the methods and devices of obtaining plethysmographic waveform or pulsatile data described herein. Judgments made using the methods and devices of this invention may also include recommendations of medical treatment or monitoring that do not require admission to a hospital as well as recommendations of admittance to a hospital and related treatments and monitoring.

### EXAMPLES

### Example 1

### Accuracy of Pulsus Paradoxus and Physician Scoring in Prediction of Subject Disposition

The methods and devices of the invention utilize measurements of pulsus paradoxus in making diagnoses of respiratory distress in a subject, sometimes in combination with physician assessments. The accuracy of pulsus paradoxus and physician scoring in correctly identifying subjects in need of admission to a hospital was evaluated.

Using the discharge and admission/relapse results as the gold standard, the sensitivity, specificity, and positive and negative predictive values of AT-PP and physician scoring were calculated. The exact binomial confidence intervals were computed for each estimate. The measure of agreement between physician and AT-PP determined disposition was computed from Cohen's Kappa statistic. All analyses were conducted with SAS VER 9.1®, the freely distributed “intracc” SAS macro (Hamer, R. H., SAS macro, Virginia Commonwealth University, ©1990), and custom functions developed internally for MatLab 7.01®, and an alpha level of 0.05 was deemed statistically significant unless otherwise noted. In addition, receiver operating curves were constructed for AT-PP as a continuous variable for prediction of admission status using pre- and post-treatment values. The area under the curve and 95% CI was computed as the c statistic by the method of Delong et al. (Biometrics, 44:837-845, 1988) in estimating the overall ability of pulsus paradoxus to distinguish between subjects who were admitted/relapsed and those whom were discharged. An optimized cutoff AT-PP threshold was selected based on optimized sensitivity and specificity, where sensitivity and specificity were equal.

All variable distributions were assessed for violation of the assumption of normality based on skewness, the Shapiro-Wilk statistic (alpha=0.01), and visualization. Variables having a significant deviation from normal via the Shapiro-Wilk statistic were submitted to three linear transformations: square root, natural logarithm, and inverse. The linear transformation that improved the distribution the most was selected. In addition, both the untransformed and transformed distributions were visually inspected to verify normality.

Seventy-nine subjects were enrolled in this study from September 2003 to June 2005 as a convenience sample. Nine subjects were excluded from the analysis as they failed to meet study asthma criteria following post-hoc inspection of both outpatient and inpatient records. Of the remaining 70 subjects, 19 (27.1%) were admitted from the emergency department. Three subjects relapsed within 72 hrs after discharge and sought medical care. Thus, 48 subjects (68.6%) had a good outcome and 22 (31.4%) had a poor outcome. The median length of stay for admitted subjects was two days. pulsus paradoxus was successfully acquired from 63 subjects during their treatment in the emergency department. Failure to acquire continuous blood pressure data occurred in 7 subjects, resulting in no AT-PP values for these subjects. Further analysis was conducted on these 63 subjects. The demographic information comparing admitted with discharged subjects is illustrated in Table 1, which shows no significant differences in gender, smoking and pulse rate. However, the admitted subjects do display statistically higher AT-PP values after treatment as illustrated in Table 1. Admitted subjects also display higher respiratory rates pre- and post-treatment compared to discharged subjects and lower values of SpO₂ post-treatment. Admitted subjects were older than discharged subjects. A significant difference in post-treatment AT-PP was observed between discharged and admitted subjects.

TABLE 1
ED Patient demographics and vital signs.

| | Discharged | Admitted† | Statistic | p |
|---|---|---|---|---|
| Gender-Female | 24 (53%) | 7 (39%) | X² = 1.073 | 0.300 |
| (%) | | | | |
| Mean Age | 38.8 (12.0) | 51.8 (22.2) | t (19.6) = 2.30 | 0.033‡ |
| in Years (SD) | | | | |
| History of | 20 (44%) | 7 (39%) | X² = 0.162 | 0.687 |
| Smoking (%) | | | | |
| | Mean (SD) | Mean (SD) | Statistic | p |
| Pre-TX Respir- | 20.1 (5.3) | 25.3 (8.4) | t (22.5) = 2.44 | 0.023‡ |
| atory Rate | | | | |
| Post-TX Respir- | 20.1 (4.2) | 25.5 (8.4) | t (20.7) = 2.60 | 0.017‡ |
| atory Rate | | | | |
| Pre-TX Pulse | 96% (3%) | 95% (5%) | t (20.1) = 1.13 | 0.270‡ |
| Oximetry | | | | |
| Post-TX Pulse | 97% (2%) | 94% (4%) | t (21.6) = 2.78 | 0.011‡ |
| Oximetry | | | | |
| Pulse Rate | 102.0 (10.1) | 97.8 (8.5) | t (59) = 1.62 | 0.111 |
| Pre-TX AT-PP | 11.5 (7.2) | 13.2 (7.4) | t (53) = 0.64 | 0.528* |
| Post-TX AT-PP | 9.1 (6.0) | 17.6 (8.4) | t (61) = 4.40 | <.001* |

*Raw Mean and SD presented, but t-test based on natural log transformed scores.
†Includes relapsed patients.
‡Satterthwaite adjustment for unequal variance applied to t-test.

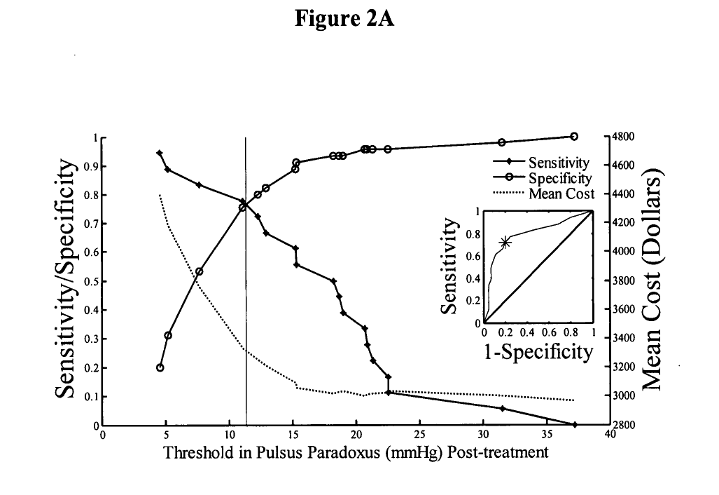

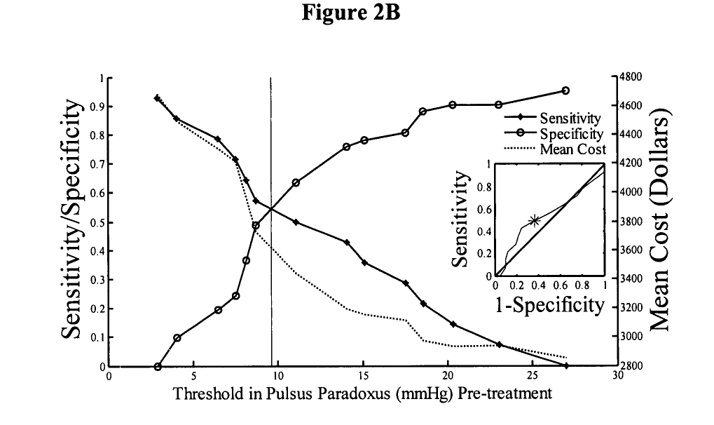

Signal detection theory-based analysis of the sensitivity and specificity of AT-PP in arriving at the discharge/admit disposition was significant for the pulsus paradoxus measurement at time 60 minutes following standardized asthma treatment. The pulsus paradoxus threshold, which maximized sensitivity and specificity, was 11.3 mmHg (FIG. 2A). The mean Wilcoxon AUROC (95% CI) was 0.82 (0.69-0.99) (FIG. 2A, inset). The risk ratio was 5.32 for admission among subjects with pulsus paradoxus, which exceeded this threshold. This is in contrast to the same analysis for the initial AT-PP measurement prior to standardized asthma treatment, in which the mean Wilcoxon AUROC (95% CI) was 0.571 (0.27-0.87) (FIG. 2B, inset). The AT-PP threshold which maximized sensitivity and specificity was 9.6 mmHg, subjects' AT-PP above this threshold had relative risk of 1.20 for admission.

Measurement of PP, embedded and automated in a continuous non-invasive blood pressure recorder (AT-PP), discriminated admitted/relapsed from discharged asthmatic adult patients and was a well tolerated procedure. The optimized AT-PP threshold for admission was >11.3 mmHg following standardized treatment. This observed threshold also compares favorably to the first NAEPP Asthma Guidelines which recommended hospital admission at a PP of 12 mmHg. The subsequent NAEPP Guidelines continue to recommend PP measurement.

The specificity and sensitivity of the physician assessments in appropriately managing asthma in this study was 0.89 and 0.83 respectively (Table 2). There were eight cases where physician management appeared correct upon audit but the automated-pulsus paradoxus (AT-PP) values failed to indicate a correct subject disposition. The specificity and sensitivity of AT-PP in appropriately managing asthma in this study was 0.78 and 0.78 respectively. The overall accuracy of AT-PP and physician disposition was 0.78 and 0.87 respectively. Interestingly, there were only two overlapping cases where inappropriate dispositions by both physicians and AT-PP occurred, suggesting each may have their relative strengths and a combinatory approach would prove better than either alone. This is also supported by the Kappa statistic which shows incomplete overlap between AT-PP and physician disposition (Table 2). A total of five subjects who were admitted may have been admitted unnecessarily judging from an audit of the inpatient medical records. These records indicate treatment for asthma but at an intensity level which could have been accomplished on an outsubject basis. In each case the length of the admission was for one day. The mean (95% CI) AT-PP measurement post-treatment for these subjects was 6.0 mmHg (2.6-9.5) compared to 17.6 mmHg (13.5-21.8) for the remaining appropriately admitted subjects (Student's t=2.95; p=0.007). A total of three subjects relapsed; two of these subjects had post-treatment AT-PP values of 21.3 and 20.7 mmHg. The mean (95% CI) AT-PP measurement for all appropriately discharged subjects was 9.1 mmHg (7.3-10.5) and was significantly different from the appropriately admitted subjects (Students's t=4.51; p<0.001). Assuming the AT-PP threshold of 11.3 mmHg was adhered to in a prospective manner, pulsus paradoxus measurement may have prevented five unnecessary admissions and two inappropriate discharges.

TABLE 2
Comparison of automated pulsus paradoxus and treating
physician-assessed disposition to patient chart audit and each other.

| | Patient Chart Audit | | | Patient Chart Audit | |
|---|---|---|---|---|---|
| AT-PP | Admitted† | Discharged | Physician | Admitted† | Discharged |
| >11.3* | 14 | 10 | Admit | 15 | 5 |
| ≦11.3 | 4 | 35 | Discharge | 3 | 40 |

| | Est. (95% CI) | | Est. (95% CI) |
|---|---|---|---|
| Sensitivity | 0.78 (0.68-0.88) | Sensitivity | 0.83 (0.74-0.93) |
| Specificity | 0.78 (0.68-0.88) | Specificity | 0.89 (0.81-0.97) |
| PPV | 0.58 (0.46-0.71) | PPV | 0.75 (0.64-0.86) |
| NPV | 0.90 (0.82-0.97) | NPV | 0.93 (0.87-0.99) |
| Accuracy | 0.78 (0.68-0.88) | Accuracy | 0.87 (0.79-0.96) |

Physician

| AT-PP | Admitted | Discharged |
|---|---|---|
| >11.3* | 13 | 11 |
| ≦11.3 | 7 | 32 |

| | Est. (95% CI) |
|---|---|
| Cohen's Kappa | 0.37 (0.14-0.61) |

*PP decision uses threshold from ROC curves (rule: >11.3 = Admit)
†Includes relapsed patients.

### Example 2

### Inter-Rater Reliability of Physician Analog Scales and Relationship Between Objective Scoring and Pulsus Paradoxus

The methods and devices of the invention utilize measurements of pulsus paradoxus in making diagnoses of respiratory distress in a subject, sometimes in combination with physician assessments. The error of pulsus paradoxus measures and physician scoring was found to be non-overlapping suggesting a combination of both methods may make a better diagnosis.

The inter-rater reliability of the objective scoring composite and sub-scales (transformed where necessary) was estimated using the intra-class correlation coefficients (ICC) as described by Shrout and Fleiss (Psychological Bulletin, 86:420-428, 1979). A mixed model was used, with “rater” treated as a random variable since each subject was rated by a pair of physicians pulled from a sample of possible physicians (the same two physicians were not always used for each subject, though the same two were used for both pre- and post-treatment time points within a given subject). The ICC of the raters was used as an index of reliability of actual rater judgments. The estimated ICC of the mean of the two raters (n=2) was used throughout the analysis.

For objective scoring measures (composite and subscales) that met or exceeded an ICC of 0.80 for the mean of the ratings at both time points, the mean of the two raters for each subject was assessed for its relationship to AT-PP using a repeated measures (pre/post treatment) general linear model with the score (continuous) as a fixed effect. In addition, each objective scoring measure (including those that failed to meet the ICC criterion) was evaluated for predicting AT-PP using hierarchical linear models (PROC MIXED SAS VER 9.1®) to assess whether or not on average there was a relationship between observer ratings and AT-PP (mean slope within rater). Residuals were examined for systematic deviations and overall model fit and scatter-plots examined to verify and assist in interpreting model parameters.

The inter-rater reliabilities as assessed with intra-class correlation are listed in Table 3. Neither the composite nor any of the sub scales met our criterion for reliability (0.80). However, the estimated mean of the composite score did meet our criterion, as well as the mean for objective dyspnea (OD) at pre-treatment. The mean total score, the only measure which met our criteria for reliability, was marginally predictive of AT-PP (Table 3), indicating that higher means generally predicted higher AT-PP. Examination using hierarchical linear modeling further revealed that while physicians did not show agreement in their absolute scores as measured by ICC, their composite and one sub-score (OD) did significantly relate to AT-PP as indicated by a significant mean slope (Table 4). This indicates that physicians agree on perceived changes in a composite assessment and OD which are correlated to changes in AT-PP. This was almost also true for prolonged expiratory phase (PEP).

TABLE 3
Inter-rater reliability of objective scoring

| | Inter-Rater | | Est. Reliability | |
|---|---|---|---|---|
| | Reliability | | of Mean | |
| | Pre-TX | Post-TX | Pre-TX | Post-TX |
| Scale (transformation) | ICC | ICC | ICC | ICC |
| Total (sqrt) | 0.732 | 0.692 | 0.845* | 0.818* |
| Objective Dyspnea (sqrt) | 0.697 | 0.586 | 0.821* | 0.739 |
| Sternocleidomastoid Muscle | 0.543 | 0.415 | 0.704 | 0.587 |
| Use (inv) | | | | |
| Prolonged Expiratory Phase (sqrt) | 0.595 | 0.611 | 0.746 | 0.758 |
| Respiratory Rate (sqrt) | 0.607 | 0.575 | 0.756 | 0.730 |
| Heart Rate (sqrt) | 0.574 | 0.729 | 0.597 | 0.747 |
| Accessory Muscle Use (log) | 0.658 | 0.538 | 0.794 | 0.699 |
| Air Entry (log) | 0.110 | 0.422 | 0.198 | 0.593 |
| Work of Breathing (log) | 0.534 | 0.581 | 0.697 | 0.735 |
| Mental Status (inv) | 0.328 | 0.557 | 0.493 | 0.715 |
| Cerebral Function (inv) | 0.278 | 0.328 | 0.435 | 0.494 |

*meets or exceeds .80 cut-off for reliability

TABLE 4
Bivariate relationships between objective scoring and automated pulsus paradoxus.

| Repeated Measures ANOVA | | SE | Df | t | p (1-tailed) |
|---|---|---|---|---|---|
| | Slope | | | | |
| Mean Total (sqrt) | 0.5161 | 0.2685 | 41 | 1.922 | 0.031 |
| | Mean Slope | | | | |
| Total (sqrt) | 0.156 | 0.053 | 34 | 2.930 | 0.003* |
| Objective Dyspnea (sqrt) | 0.228 | 0.078 | 32 | 2.902 | 0.003* |
| Sternocleidomastoid Muscle Use (inv) | 0.270 | 0.180 | 32 | 1.501 | 0.072 |
| Prolonged Exiratory Phase (sqrt) | 0.214 | 0.079 | 32 | 2.695 | 0.006 |
| Respiratory Rate (sqrt) | 0.169 | 0.074 | 33 | 2.282 | 0.015 |
| Heart Rate (sqrt) | 0.145 | 0.080 | 34 | 1.809 | 0.040 |
| Accessory Muscle Use (log) | 0.175 | 0.070 | 31 | 2.490 | 0.009 |
| Air Entry (log) | 0.165 | 0.069 | 31 | 2.396 | 0.011 |
| Work of Breathing (log) | 0.179 | 0.071 | 34 | 2.497 | 0.009 |
| Mental Status (inv) | 0.409 | 0.232 | 31 | 1.761 | 0.044 |
| Cerebral Function (inv) | 0.296 | 0.272 | 30 | 1.088 | 0.143 |

*alpha set to p < .005 for multiple correlated outcomes

### Example 3

### Derived vs. Observed Respiratory Rates

The methods and devices of the invention utilize measurements of pulsus paradoxus in making diagnoses of respiratory distress in a subject, which may include, as a step, an estimation of the respiratory rate of a subject.

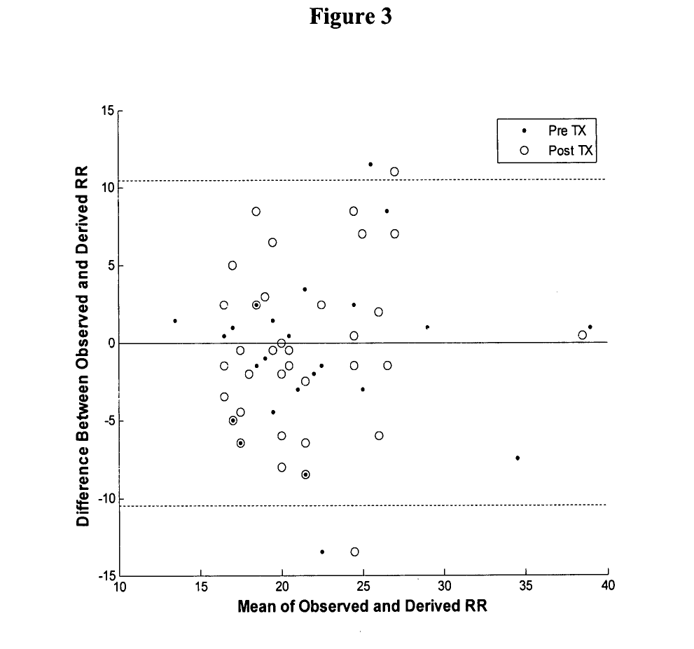

Respiratory rates from the AT-PP processing were compared to corresponding values obtained by the research assistants from direct visualization. Separate regression models were constructed for the pre- and post-treatment AT-PP measurement periods. These data were also pooled and analyzed in a Bland & Altman plot. FIG. 3 shows that the majority of both derived and observed respiratory rates fell within ±5 bpm over a range of respiratory rates from 12 to 30 bpm from both the pre- and post-treatment data sets. However, respiratory rate derived from the AT-PP monitor failed to predict those obtained by the research assistants as indicated by a lack of a significant relationship between derived and observed respiratory rate during pre-treatment: slope=0.086, intercept=21.13, F=0.199, p=0.66 and during post-treatment: slope=−0.147, intercept=24.78, F=1.178, p=0.28.

### Example 4

### Evaluation of Oximeter Plethysmography Measuring Pulsus Paradoxus (Volunteer Subject) Compared to Arterial Tomography and Transfer Functions

Various devices of the invention, referred to as cardio devices, may be used to collect pulsatile cardiorespiratory data from a subject. Two exemplary devices are an arterial tonometer and a pulse oximeter. A transfer function for measurements collected by an oximeter is described.

A change in inspiratory and expiratory plethysmographic pulse amplitude caused by pulsus paradoxus as measured by pulse oximetry was calculated for at least 10 respirations in each induced pulsus paradoxus subject and mean ±SD was calculated. The percent change in plethysmograph amplitude measured by pulse oximetry was correlated to the pulsus paradoxus measurements as obtained by arterial tomography for the same respirations and a linear regression model was constructed across the increasing degrees of negative inspiratory pressure and AT-PP. Correlation of % change in plethysmograph amplitude obtained by pulse oximetry against the AT-PP for the same respirations was performed and a linear regression model was constructed across the increasing degrees of negative inspiratory pressure and AT-PP.

Oximetry plethysmography also showed pulsus paradoxus-like phenomena, which correspond to the blood pressure measured pulsus paradoxus events (FIG. 4A). A linear regression model describes a transfer function, which relates AT-PP in units of mmHg to a decrease in plethysmographic amplitude (FIG. 4B). The slope of this relationship is roughly 0.01V/mmHg, where for each mmHg change in AT-PP, the oximeter plethysmograph peak amplitude would decrease by 0.01V. This slope of 0.01V/mmHg is an exemplary transfer function that relates a measurement in volts using an oximeter to a measurement of blood pressure in mmHg.

### Example 5

### Cost-Effectiveness Of Devices and Methods of the Invention

Cost of care was based on hospital and physician charges for outpatient and inpatient treatment of asthma. The cost of appropriate inpatient care was determined by the average level of service, cost of care per day, and average length of stay for ICD9 49390 and 49392 from inpatient billing records for 2004. This cost also included the ED charges. The cost of appropriate outpatient care was determined the same way based on one ED visit without patient relapse, which was defined as an unscheduled medical office or ED visit within 72 hrs of discharge. The cost of inappropriate inpatient care was based on the average cost in 2004 for ICD 49390 and 49392 for a 1-day admission. This cost also included the ED charges. These patients were identified in the study cohort as those patients who received a level of care which was low and could have been rendered as an outpatient. The cost of inappropriate outpatient care was based on the average 2004 costs for the initial ED visit and the cost of appropriate inpatient care described above (which includes the second ED visit). Costs of inappropriate outpatient care do not contain actuarial costs associated with the hypothetical risk of death as a result of asthma mistreatment. ICD 49391 was not utilized in this analysis as status asthmaticus is an infrequent diagnosis and is non-uniformly applied by hospital billing services.

Based on cost of care estimates, the estimated mean cost per patient was assessed for each possible AT-PP threshold. This was accomplished by first estimating the costs associated with each of the four possible combinations of decision (patient AT-PP>vs.≦threshold) and outcome (admitted vs. discharged and inappropriately admitted vs. relapsed): 1) true positive=$7340, 2) true negative=$1002, 3) false positive=$3765, and 4) false negative=$7872. These four costs were multiplied by the number of patients in their matching decision/outcome combination (total cost per decision/outcome), these subsequent four values were then summed (total cost for all patients), and the sum was then divided by the total number of patients (mean cost per patient). The result produces the mean cost per patient as a function of threshold in AT-PP.

### Example 6

### Combination of Physician Assessment and AT-PP Measure Could Reduce Error in Diagnosis

The method and devices of the invention may combine measurements of pulsus paradoxus and physician assessments to making diagnoses of respiratory distress or recommendations of admission to a hospital. Sensitivity and specificity after standardized therapy in determining correct disposition were higher overall for the treating physicians than for the AT-PP measure, reconfirming the treating physician as a gold standard in asthma management studies. This is not unexpected because a physician has multiple pieces of information to make a diagnosis, but the AT-PP performs nearly as well using only one piece of information to make a diagnosis, namely pulsus paradoxus. This motivates optimism that the combination of a physician's assessment and measurements of pulsus paradoxus by devices, such as AT-PP, will outperform either method alone. Overlapping errors of the physician's assessment and AT-PP were limited to two patients, suggesting the combination of both methods could be of clinical and economic value. There were five patients admitted with normal AT-PP measures who were considered unnecessarily admitted upon subsequent medical record audit, and two released patients whom relapsed and were determined to have had high AT-PP values. The greater number of unnecessarily admitted patients may reflect the conservative approach many physicians have in the management of asthma. The alternate disposition indicated by AT-PP, supports its inclusion as an adjunct tool in patient assessment. Relapsing discharged patients are comparatively less common. As this study progresses we anticipate observing additional relapsing asthmatic patients who were inappropriately discharged which would add to the cost of care for AT-PP>20 mmHg, resulting in a cost of care curve (FIG. 2A) which looks more U-shaped. We further posit that these latter patients, whom are discharged with an AT-PP>15 mmHg, could be managed differently if a bedside PP monitor suggested that either additional ED treatment or hospitalization was needed. Similarly, the cost of asthma care among admitted patients could be decreased by a PP measure, which objectively confirms a physiologic response to therapy. The hypothetical mean cost per patient associated with dispositioning based on AT-PP prior to treatment were comparatively higher at all thresholds. This was to be expected based on the poorer ability of AT-PP to disposition patients prior to treatment compared to after treatment, since there would have been more errors overall, and errors are costlier than correct dispositions. To these ends, a device capable of measuring pulsus paradoxus could be sold in a kit with instructions for combining the physician's assessment of a subject with pulsus paradoxus measured by the device, so that a diagnosis of respiratory distress or a recommendation of admission to a hospital may be made.

The advantage of combining a physician's assessment with pulsus paradoxus measured by a device is evident given that agreement between physicians performing objective asthma scoring was lacking. For both the pre- and post-treatment periods their scores had low intraclass correlations (Table 3) and little similarity in absolute objective asthma severity scores. However, while absolute scores varied, physicians did show similar trends in ratings of some of the physical exam findings across the standardized treatment period (Table 4). Most notably objective dyspnea, and possibly prolonged expiratory phase, followed similar trends and appear to be exam findings which physicians monitor, though they rate absolute magnitude differently. Ratings in objective dyspnea also correlated with AT-PP. These results are indicative of the lack of consensus among treating physicians in rating asthma severity.

### Example 7

### Extended Monitoring of Pulsus Paradoxus

Pulsus paradoxus may be monitored over an extended period of time using the devices and methods of this invention to track a subject's medical condition over time. Like other vital signs, PP offers the opportunity to follow disease progression and response to therapy. As a unique pathophysiologic vital sign, PP can also be used as a screening vital sign on patients with undifferentiated dyspnea. The rapid evaluation for PP in ED triage could drive the differentiation of subjective dyspnea in the emergency patient population. As a group, patients with dyspnea occupy 20% of this patient population. Patients with asthma, pericardial effusions or tamponade, massive pulmonary embolus, tension pneumothorax, or severe dehydration will also manifest elevated pulsus paradoxus. Patients with silent chest asthma could be more readily identified during triage evaluation. Continuous PP monitoring also offers the opportunity to assess the response of asthma and croup to pharmacotherapy. This will also be important in evaluating new products in the management of both diseases, as PP has been used in previous pharmacologic trials. It may also become possible to remotely monitor asthma severity via continuous PP, which would benefit many patients with a well-established diagnosis. Monitoring patients in this way could avoid unnecessary ED visits and hospitalizations, which account for the largest proportion of asthma care costs. Finally, continuous PP monitoring would add a new dimension in the identification of obstructive sleep apnea by identifying upper glottis closure and pathophysiologic dyscrasias before hypoxia occurs among patients undergoing sleep studies.

The AT-PP detection algorithm for a continuous blood pressure monitor used in this study was accurate and precise, meeting Association for the Advancement of Medical Instrumentation tolerance requirements for medical devices. This algorithm should also be transferable to other continuous and non-invasive blood pressure monitors. In the event that continuous non-invasive blood pressure monitoring becomes more available in acute care settings, we believe that PP could replace PEFR as the preferred metric of acute asthma severity. PEFR alone appears to be unpredictive of patient outcome in acute asthma (Rodrigo et al., Chest 104:1325-1328, 1993) and is no longer recommended by the American College of Emergency Physicians. In a study of acute asthma in pediatric patients, PP appeared to be a surrogate for spirometry in evaluating asthma severity (Wilson et al., J. Intensive Care Med. 18:275-285, 2003). Finally, PEFR meters, which are manufactured by a number of different manufacturers, appear to have variable accuracy (Miller et al., Thorax 47:904-909, 1992).

An instrument, like a PP monitor, could serve as a patient management decision aid or in the detection of cardiopulmonary dyscrasias.

### Example 8

### Estimation of Pulsus Paradoxus of a Hypothetical Plethysmographic Waveform By Combining Period Amplitude Analysis and Power Spectrum Analysis

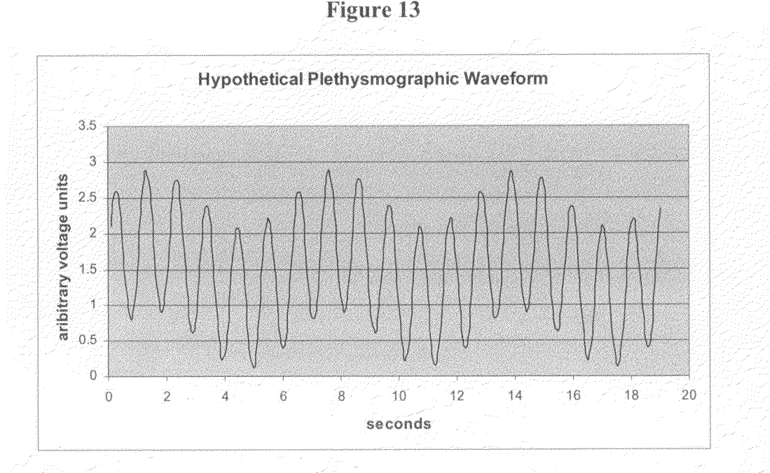

The hypothetical plethysmographic waveform depicted in FIG. 13, derived from the function f(x)=0.4 sin(x)+sin(6x)+1.5 is analyzed by two forms of waveform analysis, namely power spectrum analysis and period amplitude analysis. A hypothetical physician's assessment of a subject with this plethysmographic waveform is also shown. Measurements of pulsus paradoxus and probability (recommendations) of admission to a hospital are shown using power spectrum analysis, period amplitude analysis, and physician's assessment in the top half of the table. Notice that the estimated amplitude of sin(x) measured from the middle of the sin(x) wave (the “respiration component” of the plethysmographic waveform in power spectrum analysis) is half the estimated difference in peak heights (measured from top to bottom) obtained using period amplitude analysis: this suggests that the two forms of waveform analysis agree that the respiration component has an amplitude of ˜0.4 measured from the middle of the respiration component wave. Methods of combining the measurements of pulsus paradoxus and probabilities of admission to a hospital are shown in the bottom half of the table, e.g., averages, sums, products, extrema such as maximum and minimum, and an indication whether or not the measurements or probabilities are within 50% of each other (an exemplary reliability index): the combination of power spectrum analysis and period amplitude analysis is shown as well as their combination with a physician's assessment. Depending on the method of combining selected, different measurements of pulsus paradoxus or different recommendations of medical admission may be obtained. Some methods of combining, such as the product or sum, do not lend themselves to direct interpretation, but they can be compared to known distributions of sums or products obtained from healthy subjects or subjects experiencing respiratory distress.

TABLE 5
Methods of Combining Applied to a Hypothetical Plethysmographic Waveform

| Power Spectrum of Pleth. Waveform | Period Amplitude Analysis | Physician's Assessment |
|---|---|---|
| SIN(X) Amplitude | | |
| 0.4 | Max Peak Height in Period from 4 to 11 sec. | |
| ″Respiration Component″ | 2.88606796 | |
| SIN(6X) Amplitude | Min Peak Height: 4 to 11 | |
| 1 | 2.06 | |
| ″Pulse Component″ | Estimated Pulsus Paradoxus in Volts | |
| Estimated Pulsus Paradoxus in Volts | Max Height-Min Height | |
| 0.4 | 0.82606796 | |
| Transfer Function (mmHg/Volt) | Transfer Function (mmHg/Volt) | |
| 27 | 14 | |
| Pulsus Paradoxus | Pulsus Paradoxus | Pulsus Paradoxus |
| 10.8 | 11.56495143 | 10 |
| Probability of Admission (hypothetical) | Probability of Admission (hypothetical) | Probability of Admission (hypothetical) |
| 0.52 | 0.85 | 0.4 |
| Combining P.S. and P.A. | Pulsus Paradoxus (mmHg) | Probability of Admit to Hospital |
| Mean | 11.18247572 | 0.685 |
| Product | 124.9014755 | 0.442 |
| Sum | 22.36495143 | 1.37 |
| Max | 11.56495143 | 0.85 |
| Min | 10.8 | 0.52 |
| Within 50%? | YES | YES |
| Combining All Three | Pulsus Paradoxus (mmHg) | Probability of Admit to Hospital |
| Mean | 10.78831714 | 0.59 |
| Product | 1249.014755 | 0.1768 |
| Sum | 32.36495143 | 1.77 |
| Max | 11.56495143 | 0.85 |
| Min | 10 | 0.4 |
| Within 50%? | YES | NO |

### Example 9

### Combination of Percentage Oxygenated Hemoglobin and Pulsus Paradoxus in Diagnosing Respiratory Distress

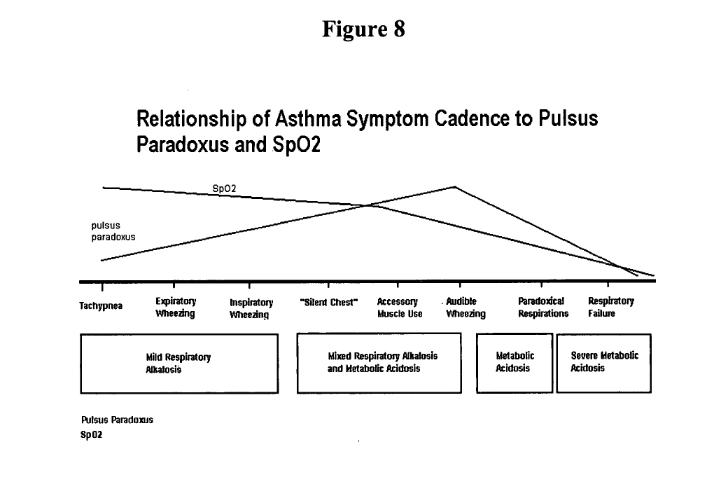

Respiratory distress may be diagnosed by the combination of measurements of percentage oxygenated hemoglobin (SpO₂) and pulsus paradoxus obtained using a pulse oximeter. Examining FIG. 8, one method of combining SpO₂ and pulsus paradoxus may entail associating each value of SpO₂ and pulsus paradoxus with a degree of respiratory distress (such as the degrees of distress implied by the ordering of the symptoms on the x-axis in increasing severity from left to right) and then taking the larger of the two degrees of respiratory distress as the final measurement. As an example, a negligible decrease in SpO₂ may be exceeded by a significant increase in pulsus paradoxus in its associated respiratory distress thus motivating a correct diagnosis of respiratory distress, potentially not detected by SpO₂. Pulsus paradoxus and SpO₂ may be combined using a PP/SpO₂ ratio such as [PP−5 mmHg]/[100−SpO2] numerically scaled to 0-1.0 where a higher number indicates worsening asthma severity before hypoxia ensues. Typically the inflection point of hypoxia is an SpO₂ of 93%. A device that combines pulsus paradoxus and SpO₂ to diagnose respiratory distress may include a pulse oximeter coupled to a digital processor (see FIG. 14 for example).

### Other Embodiments

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

What is claimed is:

1. A method for measuring pulsus paradoxus in a subject comprising:
(i) collecting pulsatile cardiorespiratory data from said subject;
(ii) performing period amplitude analysis on said data;
(iii) performing power spectrum analysis on said data; and
(iv) combining the analyses of steps (ii) and (iii) to determine a measurement for pulsus paradoxus.

2. The method of claim 1, further comprising comparing the measurement for pulsus paradoxus in said subject to that obtained in a healthy subject, wherein a determination that the measurement for said subject exceeds the measurement for said healthy subject by at least 10% indicates said subject is experiencing respiratory distress.

3. The method of claim 2, wherein said comparing yields a difference in blood pressure measured in mmHg.

4. The method of claim 1, wherein said data are presented as a plethysmographic waveform.

5. The method of claim 1, wherein said data is collected from said subject over the course of a time interval of at least 30 seconds.

6. The method of claim 5, wherein said time interval is at least 60 seconds.

7. The method of claim 6, wherein said time interval is at least 2 minutes.

8. The method of claim 4, wherein said waveform is obtained by a pulse oximeter.

9. The method of claim 4, wherein said waveform is obtained by an arterial tonometer.

10. The method of claim 4, wherein said waveform is obtained by a finometer.

11. The method of claim 1, wherein said data are filtered using a bandpass filter.

12. The method of claim 11, wherein said bandpass filter substantially excludes pulse frequencies less than 3 times the frequency of respiration of said subject or pulse frequencies greater than 7 times the frequency of respiration of said subject.

13. The method of claim 1, wherein said period amplitude analysis comprises a determination of the maximum difference in height of any two peaks, the maximum difference in area under any two peaks, the maximum difference in slope of any two peaks, or the maximum difference in curve length of any two peaks present in said data.

14. The method of claim 1, wherein said period amplitude analysis comprises a determination of the average maximum difference in height of any two peaks, the average maximum difference in area under any two peaks, the average maximum difference in slope of any two peaks, or the average maximum difference in curve length of any two peaks present in said data.

15. The method of claim 1, further comprising converting said period amplitude analysis into a change in blood pressure associated with pulsus paradoxus.

16. The method of claim 15, wherein said change is at least 10 mmHg indicating respiratory distress and motivating medical admission of a subject.

17. The method of claim 16, wherein said change is at least 11 mmHg.

18. The method of claim 17, wherein said change is at least 12 mmHg.

19. The method of claim 15, wherein said converting is performed using a transfer function determined from data of subjects experiencing respiratory distress.

20. The method of claim 19, wherein said transfer function is 0.01 Volts/mmHg.

21. The method of claim 19, wherein said respiratory distress is caused by asthma.

22. The method of claim 19, wherein said respiratory distress is created by artificial means.

23. The method of claim 1, wherein step (ii) further comprises comparing said period amplitude analysis with period amplitude analysis determined using pulsatile cardiorespiratory data from subjects experiencing respiratory distress.

24. The method of claim 23, wherein said comparing yields a difference measured in mmHg.

25. The method of claim 23, wherein said respiratory distress is caused by asthma.

26. The method of claim 23, wherein said respiratory distress is created by artificial means.

27. The method of claim 1, wherein step (ii) further comprises comparing said period amplitude analysis with period amplitude analysis determined using pulsatile cardiorespiratory data from healthy subjects.

28. The method of claim 27, wherein said comparing yields a difference measured in mmHg.

29. The method of claim 1, wherein said power spectrum analysis comprises a determination of signal amplitude associated with respiration present in said data.

30. The method of claim 1, wherein said power spectrum analysis comprises a determination of average signal amplitude associated with respiration present in said data.

31. The method of claim 1, further comprising converting said power spectrum analysis into a change in blood pressure associated with pulsus paradoxus.

32. The method of claim 31, wherein said change is at least 10 mmHg indicating respiratory distress and motivating medical admission of a subject.

33. The method of claim 32, wherein said change is at least 11 mmHg.

34. The method of claim 33, wherein said change is at least 12 mmHg.

35. The method of claim 31, wherein said converting is performed using a transfer function determined from data of subjects experiencing respiratory distress.

36. The method of claim 35, wherein said transfer function is a quadratic function.

37. The method of claim 35, wherein said respiratory distress is caused by asthma.

38. The method of claim 35, wherein said respiratory distress is created by artificial means.

39. The method of claim 1, wherein step (ii) further comprises comparing said power spectrum analysis with power spectrum analysis determined using pulsatile cardiorespiratory data from subjects experiencing respiratory distress.

40. The method of claim 39, wherein said comparing yields a difference in blood pressure measured in mmHg.

41. The method of claim 39, wherein said respiratory distress is caused by asthma.

42. The method of claim 39, wherein said respiratory distress is created by artificial means.

43. The method of claim 1, wherein step (ii) further comprises comparing said power spectrum analysis with power spectrum analysis determined using pulsatile cardiorespiratory data from healthy subjects.

44. The method of claim 43, wherein said comparing yields a difference in blood pressure measured in mmHg.

45. The method of claim 1, wherein said combining comprises converting said period amplitude analysis and said power spectrum analysis into changes in blood pressure associated with pulsus paradoxus and averaging said changes.

46. The method of claim 1, wherein said combining comprises converting said period amplitude analysis and said power spectrum analysis into changes in blood pressure associated with pulsus paradoxus and calculating a moving average of said changes.

47. The method of claim 1, wherein said combining comprises converting said period amplitude analysis and said power spectrum analysis into changes in blood pressure associated with pulsus paradoxus and calculating a Kappa statistic relating said changes.

48. The method of claim 1, wherein said combining comprises converting said period amplitude analysis and said power spectrum analysis into changes in blood pressure associated with pulsus paradoxus and calculating a test statistic that determines whether the smaller of the two said changes in blood pressure is at least 50% of the size of the larger of the two said changes in blood pressure.

49. The method of claim 1, wherein said combining comprises converting said period amplitude analysis and said power spectrum analysis into changes in blood pressure associated with pulsus paradoxus and averaging said changes, calculating a moving average of said changes, calculating a Kappa statistic relating said changes, or calculating a test statistic that determines whether the smaller of the two said changes in blood pressure is at least 50% of the size of the larger of the two said changes in blood pressure, wherein a determination that the average of said changes in blood pressure is at least 10 mmHg indicates that said subject is in respiratory distress.

50. The method of claim 49, wherein said average is at least 11 mmHg.

51. The method of claim 50, wherein said average is at least 12 mmHg.

52. The method of claim 49, wherein the average of said changes in blood pressure is between 5 mmHg and 11 mmHg, and wherein said changes motivate medical monitoring of said subject

53. A method for measuring pulsus paradoxus in a subject comprising:
(i) collecting pulsatile cardiorespiratory data from said subject;
(ii) performing a first form of waveform analysis on said data;
(iii) performing a second form of waveform analysis on said data; and
(iv) combining the analyses of steps (ii) and (iii) to determine a measurement for pulsus paradoxus.

54. The method of claim 53, wherein step (iv) further comprises combining a third form of waveform analysis performed on said data with said first and said second forms to measure pulsus paradoxus.

55. A device for measuring pulsus paradoxus in a subject comprising:
(i) an optical plethysmograph to collect pulsatile cardiorespiratory data from said subject;
(ii) a processor to perform period amplitude analysis on said data;
(iii) a processor to perform power spectrum analysis on said data; and
(iv) a processor to combine the analyses of steps (ii) and (iii) to determine a measurement for pulsus paradoxus.

56. The device of claim 55, further comprising a bandpass filter to filter said data.

57. The device of claim 55, wherein said bandpass filter substantially excludes pulse frequencies less than 3 times the frequency of respiration of said subject or pulse frequencies greater than 7 times the frequency of respiration of said subject.

58. A device for measuring pulsus paradoxus in a subject comprising:
(i) an arterial tonometer to collect pulsatile cardiorespiratory data from said subject;
(ii) a processor to perform period amplitude analysis on said data;
(iii) a processor to perform power spectrum analysis on said data; and
(iv) a processor to combine the analyses of steps (ii) and (iii) to determine a measurement for pulsus paradoxus.

59. The device of claim 58, further comprising a bandpass filter to filter said data.

60. The device of claim 58, wherein said bandpass filter substantially excludes pulse frequencies less than 3 times the frequency of respiration of said subject or pulse frequencies greater than 7 times the frequency of respiration of said subject.

61. A device for measuring pulsus paradoxus in a subject comprising:
(i) a finometer to collect pulsatile cardiorespiratory data from said subject;
(ii) a processor to perform period amplitude analysis on said data;
(iii) a processor to perform power spectrum analysis on said data; and
(iv) a processor to combine the analyses of steps (ii) and (iii) to determine a measurement for pulsus paradoxus.

62. The device of claim 61, further comprising a bandpass filter to filter said data.

63. The device of claim 61, wherein said bandpass filter substantially excludes pulse frequencies less than 3 times the frequency of respiration of said subject or pulse frequencies greater than 7 times the frequency of respiration of said subject.

64. A device for measuring respiratory distress in a subject comprising:
(i) a optical plethysmograph to collect pulsatile cardiorespiratory data from said subject;
(ii) a processor to calculate pulsus paradoxus from said data;
(iii) a processor to calculate percentage oxygenated hemoglobin from said data; and
(iv) a processor to combine the output of steps (ii) and (iii) to determine a measurement of respiratory distress.

65. A method for measuring respiratory distress in a subject comprising:
(i) collecting pulsatile cardiorespiratory data from said subject;
(ii) estimating pulsus paradoxus using said data;
(iii) estimating the percentage of hemoglobin (Hb) which is saturated with oxygen; and;
(iv) combining the analyses of steps (ii) and (iii) to determine a measurement of respiratory distress.

66. The device of claim 55, wherein said optical plethysmograph is a pulse oximeter.

67. The device of claim 64, wherein said optical plethysmograph is a pulse oximeter.
